(19) United States
(12) Patent Application Publication
(10) Pub. No.: US 2008/0109914 A1
(43) Pub. Date: May 8, 2008

(54) DIFFERENTIAL LABELING OF CELLS

(76) Inventors: Brian Popko, Chicago, IL (US); Roumen Balabanov, Chicago, IL (US); Trent Watkins, Palo Alto, CA (US)

Correspondence Address: WILSON SONSINI GOODRICH & ROSATI, 650 PAGE MILL ROAD, PALO ALTO, CA, 94304-1050, US

(21) Appl. No.: 11/837,437

(22) Filed: Aug. 10, 2007

Related U.S. Application Data
(60) Provisional application No. 60/822,001, filed on Aug. 10, 2006.

(51) Int. Cl. A01K 67/00; C12Q 1/68; C12N 15/00; C12N 5/06

(52) U.S. Cl. 800/3; 435/6; 435/320.1; 435/325; 800/13; 800/9

(57) ABSTRACT

The invention relates to the generation of an animal model that exhibits neural cell-specific expression of a marker gene that correlates to remyelination or myelin repair. The compositions and methods embodied in the present invention are particularly useful for drug screening and/or treatment of demyelination disorders, particularly in identifying compounds that promote or inhibit remyelination.

### CROSS REFERENCE

This application claims the benefit of U.S. Provisional Application No. 60/822,001, filed Aug. 10, 2006, which is incorporated herein by reference in its entirety.

### BACKGROUND OF THE INVENTION

Neuronal demyelination is a deleterious condition characterized by a reduction of myelin in the nervous system. Myelin is a vital component of the central (CNS) and peripheral (PNS) nervous system, which encases the axons of neurons and forms an insulating layer known as the myelin sheath. The presence of the myelin sheath enhances the speed and integrity of nerve signals in the form of electric potentials propagating down the axon. The loss of myelin sheath produces significant impairment in sensory, motor and other types of functioning as nerve signals reach their targets either too slowly, asynchronously (for example, when some axons in a nerve conduct faster than others), intermittently (for example, when conduction is impaired only at high frequencies), or not at all.

Neural tissue comprises neurons and supporting or glial cells. Glial cells outnumber neurons by about ten to one in the mammalian brain. Glial cells may be divided into four types: astrocytes, oligodendrocytes, Schwann cells and microglial cells. The myelin sheath is formed by the plasma membrane or plasmalemma of a type of glial cell, namely oligodendrocytes in the CNS, and Schwann cells in the PNS. During the active phase of myelination, each oligodendrocyte in the CNS must produce as much as approximately 5000 μm² of myelin surface area per day and approximately 10⁵ myelin protein molecules per minute (Pfeiffer et al., Trends Cell Biol. 3:191-197 (1993)). Myelinating oligodendrocytes have been identified at demyelinated lesions, indicating that demyelinated axons may be repaired with the newly synthesized myelin.

Neuronal demyelination is manifested in a large number of hereditary and acquired disorders of the CNS and PNS. These disorders include Multiple Sclerosis (MS), Progressive Multifocal Leukoencephalopathy (PML), Encephalomyelitis, Central Pontine Myelolysis (CPM), Anti-MAG Disease, Leukodystrophies, Adrenoleukodystrophy (ALD), Alexander's Disease, Canavan Disease, Krabbe Disease, Metachromatic Leukodystrophy (MLD), Pelizaeus-Merzbacher Disease, Refsum Disease, Cockayne Syndrome, Van der Knapp Syndrome, and Zellweger Syndrome, Guillain-Barre Syndrome (GBS), chronic inflammatory demyelinating polyneuropathy (CIDP), and multifocual motor neuropathy (MMN). For the vast majority of these disorders, there are no cures and few effective therapies.

Multiple sclerosis is the most common demyelinating disease of the central nervous system, affecting approximately 6,000,000 people worldwide and some 250,000 to 350,000 people in the United States. The disease is characterized clinically by relapses and remissions, leading eventually to chronic disability. The earlier phase of multiple sclerosis is characterized by the autoimmune inflammatory strike against myelin sheath leading to paralysis, lack of coordination, sensory disturbances and visual impairment. The subsequent chronic progressive phase of the disease is typically due to active degeneration of the myelin sheath and inadequate remyelination of the demyclinated lesions (Franklin, Nat. Rev. Neurosci. 3:705-714 (2002); Bruck et al., J. Neurol. Sci. 206.181-185 (2003); Compston et al., Lancet 359.1221-1231 (2002)).

It is known that oligodendrocytes are the principal target cells of demyelinating disorders and that recovery from these disorders necessitates the restoration of the normal myelin by oligodendrocytes. However, remyelination is often an inefficient process leading to significant disability and/or death. Evidence is accumulating that the principal cellular mechanisms of remyelination may differ with developmental myelination (Franklin, Nat. Rev. Neurosci. 3.705-714 (2002); Balabanov et al., Nat. Neurosci. 8.262-264 (2005), Farhadi et al., J. Neurosci. 23:10214-10223 (2003); Ruffini et al., Am. J. Pathol. 165: 2167-2175 (2004); Arnett et al., Science 306:2111-2115 (2004); Stidworhty et al., Brain 127: 1928-1941 (2004)). Therefore understanding remyelination is one of the key aspects of identifying cause, effects and ameliorative options for myelin repair. Indeed, a major challenge in MS research is to understand the cause of remyelination failure and to devise ways of ameliorating its consequences. In recent years, several lines of evidence have suggested that the demyelinated lesions in MS are not deficient in oligodendrocyte progenitor cells (OPCs), rather that remyelination failure is associated with the insufficient repopulation of oligodendrocytes (Chang et al., J. Neurosci. 20: 6404-6412 (2000), Lucchinetti et al., Brain 122:2279-2295 (1999); Maeda et al., Ann. Neurol. 49.776-785 (2001)).

Currently, the ability to identify remyelinated axons is based on the premise that the myelin sheath of such axons tends to be thinner, which requires electron microscopy (EM) analysis for identification. EM analysis is prohibitively arduous and costly for the routine analysis of in vivo remyelination, especially in situations such as experimental autoimmune encephalomyelitis (EAE), where the demyelination and remyelination may not be precisely localized to one particular locus. Therefore, there remains a considerable need for methods and compositions that can facilitate the identification and elucidation of the molecular basis of neuronal remyelination. There also exists a pressing need for developing biologically active agents effective for promoting remyelination, as well as identifying agents that may inhibit remyelination. The same applies to neuronal injury.

Neuronal injury is a cause for numerous deleterious conditions of the nervous system. Neurons in the CNS have poor regenerative capacity and thus, injury to the CNS often results in functional impairments that are largely irreversible. Damage resulting from stroke, trauma, or other causes can result in life-long losses in cognitive, sensory and motor functions, and even maintenance of vital functions. Numerous diseases, such as Alzheimer's disease, Parkinson's disease, stroke, head and spinal cord trauma, are all associated with damage to the CNS that is often severe, long lasting, or even permanent.

Neuronal cells that are lost are usually not replaced, and those that are spared are generally unable to re-grow severed connections, although a limited amount of local synaptic reorganization can occur close to the site of injury. Regenerative failure in the CNS has been attributed to a number of factors, which include the presence of inhibitory molecules on the surface of glial cells that suppress axonal growth, absence of appropriate substrate molecules such as laminin to foster growth, and an absence of the appropriate trophic factors needed to activate programs of gene expression required for cell survival and differentiation.

Neurons in the PNS have a relatively higher regenerative capacity. See, for example, Horner & Gage, Nature 407:963 970 (2000). Injured nerve fibers can re-grow over long distances, with eventual excellent recovery of function. It has been reported that the difference in regenerative capacity is not due to intrinsic difference, for example, neurons of the CNS will extend their axons over great distances if given the opportunity to grow through a grafted segment of PNS (e.g., sciatic nerve). Therefore, neurons of the CNS are believed to retain a capacity to grow if given signals promoting regrowth from the extracellular environment. It is thought that the ability of neurons to regenerate an axon after injury is determined by intrinsic factors of the damaged neuron and the surrounding environment. Despite extensive research efforts, the progress in elucidating the precise molecular mechanism involving neuronal regeneration has been hampered by the lack of a convenient research tool and model to ascertain and quantify neuronal regeneration. As such, there remains a considerable need for methods and compositions that will aid in the detection and quantification of neuronal regeneration.

The present invention satisfies these needs and provides related advantages as well.

### SUMMARY OF THE INVENTION

The present invention provides a method for distinguishing pre-existing myelinating cells from remyelinating cells upon a demyelinating insult. The method comprises the steps of (a) introducing into a population of neural cells a plurality of transgenes, wherein at least a first transgene encodes a first fluorescent marker protein and a second transgene encodes a second fluorescent marker protein, wherein said first marker protein and said second marker protein emit different detectable wavelengths; and wherein expression of said first transgene is indicative of pre-existing myelinating cells prior to induction of demyelination, and expression of said second transgene is indicative of remyelinating cells; (b) subjecting said population of neural cells to a demyelinating insult; and (c) identifying cells expressing said first or said second marker protein, thereby distinguishing said pre-existing myelinating cells from said remyelinating cells. The subject method may further comprise the step of quantifying the extent of remyelination by counting the number of cells expressing the second marker protein. In one aspect, the first transgene and the second transgene are designed to be operably linked to a regulatory element specific for myelinating cells. In a preferred aspect, the second transgene is temporally controlled by a third transgene expressed in a progenitor cell that exhibits capability to remyelinate upon a demyelinating insult. In yet a preferred aspect, the second transgene is expressed when expression of said first transgene is suppressed, which can be carried out by excising the first transgene from an expression operon.

The present invention also provides a method of distinguishing pre-existing neurons from regenerated neurons upon a neural damage. The method comprises (a) introducing into a population of neuronal cells a plurality of transgenes, wherein at least a first transgene encodes a first fluorescent marker protein and a second transgene encodes a second fluorescent marker protein, wherein said first marker protein and said second marker protein emit different detectable wavelengths; and wherein expression of said first transgene is indicative of pre-existing neurons prior to a neural damage, and expression of said second transgene is indicative of regenerated neurons; (b) subjecting said population of neuronal cells to a neural damage; and (c) identifying neuronal cells expressing said first or said second marker protein, thereby distinguishing said pre-existing neurons from said regenerated neurons. In one aspect, the second transgene is expressed when expression of the first transgene is suppressed. In another aspect, expression of the second transgene is temporally controlled by a third transgene, wherein the third transgene is operably linked to a regulatory element inducible upon axonal damage, and wherein upon expression of the third transgene, expression of the first transgene is suppressed.

The present invention provides compositions and methods designed to provide a model system for detecting and quantifying remyelination. In one aspect, the invention provides a transgenic animal comprising a transgene encoding a marker protein whose expression is inducible via an exogenous agent, whereby expression is controlled by a regulatory element from a gene that is differentially expressed in a glial cell, and where expression of the marker protein occurs differentially in a subpopulation of specific glial cells.

The subpopulation of glial cells are mature or progenitor oligodendrocytes. Furthermore, the subpopulation of cells can be remyelinating and/or myelinating. In addition, the regulatory element is selected from any gene that is differentially expressed in glial cells, particularly glial cells involved in myelination or remyelination. In other embodiments, the subpopulation of glial cells are Schwann cells, astrocytes, neurons, or axons; the cells may be mature or progenitor cells.

In another aspect, the present invention provides a transgenic animal comprising at least two transgenes encoding two or more marker proteins, wherein expression of one or more marker proteins is temporally controlled by an exogenous agent, wherein expression occurs differentially in two different subpopulations of glial cells. In one example, the marker proteins are fluorescent marker proteins, each emitting a different detectable wave length. Furthermore, detection of different marker proteins is indicative of the presence or absence of remyelinating neural cells in the animal's nervous system.

In addition, the present invention provides cells that are obtained from the transgenic animals of the invention, which cells are utilized in in vitro or in vivo methods, such as in cell culture methods. In addition, the present invention provides vectors that are suitable for in vivo and in vitro use, such as in cell culture or transgenic use.

The transgenic animals, cells and vectors of the present invention provide for a model system to detect, or detect and quantify, as well as distinguish, myelination from remyelination. The model system comprises designing two different transgenic animals, which are subsequently combined to produce a double transgenic animal, an overview for which process is illustrated in FIGS. 2 and 3.

In one aspect of the invention, a first transgenic animal is designed to express a cognate recombinase protein that exploits the transcriptional control region from the gene sequence that operates in a cell-specific manner. In addition, the recombinase is engineered to be active only when induced, for example, by an exogenous agent.

In another aspect of the invention, a second transgenic animal is designed to express a reporter gene/product that exploits the transcriptional control region from the gene sequence that operates in a cell-specific manner. In one embodiment, the gene construct to be introduced comprises two genes of interest, in combination with a promoter/enhancer element form one transcriptional unit. However, the first gene of interest comprises a transcription termination signal (e.g., stop codon) and further the gene is flanked by sequences capable of recognition by a cognate recombinase protein (e.g Cre). Furthermore, operably linked to this gene is the second gene of interest that is only expressed if the first gene of interest is excised, where such excision can be mediated in the presence of a recombinase protein recognizing the cognate flanking sequences. In addition, the proteins of interest are designed so that expression includes a cell localization signal that localizes the proteins of interest to the desired cell membrane. Therefore, the resulting transgenic animal is capable of producing a first or second desired gene product, each in a cell-specific manner, and where expression of the second gene of interest is dependant on a site-specific recombination excision of the first gene of interest.

In a further aspect of the present invention, the first and second transgenic animals described above are combined (i.e., mated) and progeny is selected comprising the transgenes of the first and second animals, the inducible recombinase and the reporter genes. The progeny transgenic animal of the invention will provide a system to identify and quantify remyelination. In one embodiment, a molecule is administered to the transgenic animal to functionally activate the recombinase protein in a cell-specific manner. Furthermore, the functional recombinase expression allows for excision of the first gene of interest and thus expression of the second gene of interest in a cell-specific manner. Therefore, the progeny transgenic animal will express an inducible form of the recombinase protein in a cell specific manner. The recombinase can excise the first reporter gene, thereby allowing expression of the second gene of interest in a cell-specific manner. In a preferred embodiment, the first and second genes of interest encode fluorescence proteins, as further described herein.

In a preferred embodiment, the selected progeny transgenic animals will express the first gene of interest in one cell type and the second gene of interest in a second cell type. Therefore, in monitoring expression of the genes of interest, the model system allows identification and/or quantification of cell-specific expression of a particular gene of interest, and to distinguish whether such expression correlates to remyelination versus myelination. In the transgenic animal, where a recombinase is controllably expressed and the first gene of interest is subsequently excised, the model system allows for specific detection and quantification of the second gene of interest which can correlate to remyelination.

In one embodiment, the genetic construct used to obtain a first transgenic animal encodes a platelet derived growth factor-α (PDGFα) receptor gene operably linked to a recombinase gene, where the recombinase is functionally controlled (induced) by an exogenous agent, such as CreER^{t2}. The promoter will be captured from the endogenous PDGFα receptor gene. The recombinase is inducible in so far as following administration of a synthetic steroid hormone (tamoxifen), the CreER^{t2} protein translocates into the nucleus where it is functional. In addition, a second genetic construct is used to obtain a second genetic animal, where the construct encodes a first marker gene that is “floxed” and a second marker gene downstream of the first marker gene, where both genes are under control of a proteolipid protein (PLP) promoter element. The resulting transgenic animals are subsequently mated and progeny are genotyped to identify animals carrying the first and second gene constructs. As such, a preferred progeny is a double transgenic (F1) used for assays of the present invention.

Accordingly, in one aspect of the present invention, a transgenic animal model system is provided free of the artifacts of tissue culture to assay remyelination. Furthermore, in certain aspects of the invention, a transgenic animal's nervous system is subjected to physical and neurotoxic challenges, either through an invasive or non-invasive procedure, where the model system comprising such a transgenic animal also provides a means for detection of neurologic repair or toxicity. Furthermore, the model system allows monitoring effects of challenges or effects of administration of biologically active agents over a period of time in a transgenic animal. Furthermore, such monitoring effects can be on more than one occasion in the same animal.

In another aspect, the present invention provides a non-human transgenic animal having: (a) stably integrated into the genome of said animal a transgenic nucleotide sequence encoding one or more reporter genes or reporter proteins; and (b) capability of providing neural cell-specific expression of said one or more reporter genes or reporter proteins each of which is cell-specifically expressed in an animal.

In addition, the present invention comprises cells derived from the subject transgenic animals. Furthermore, such cells can be further genetically modified for use in cell culturing techniques and cell-based assays to study biochemical, biopharmaceutical, or myelin formation mechanistic phenomena.

The present invention also provides methods of producing transgenic animals that provide a system for identifying a candidate biological agent that promotes remyelination. The method comprises (a) constructing gene constructs that are capable of differentially expressing one or more genes or gene products that encode reporter proteins; (b) introducing said gene constructs into one or more animals; (c) detecting expression or modulation of expression of said one or more reporter proteins; and (d) determining based on expression of one or more reporter proteins whether neuronal myelination and/or remyelination occurs in response to administration of said agent.

In a related but separate embodiment, the present invention provides a method for determining whether remyelination occurs in an animal. The method comprises the steps of (a) providing a subject transgenic animal; (b) administering said exogenous agent to induce expression of said third transgene; (c) subjecting said animal to a demyelinating insult; and (d) detecting expression of said first and/or said second marker protein, thereby determining whether remyelination has occurred.

The present invention also provides an assay where a biologically active agent is administered to one animal (i.e., test animal) to compare differential cell-specific expression of marker genes/gene products as compared to a control animal (i.e., agent not administered), where the marker gene product is detected/quantified so as to determine if administration of the agent results in modulation of remyelination.

The present invention further provides a method of developing a biologically active agent that promotes neuronal remyelination utilizing cell culture assays. The method comprises (a) obtaining and culturing neural cells from the transgenic animals produced by methods described herein; (b) contacting a candidate biologically active agent with a myelinating cell from a demyelinated lesion of a subject; and (b) detecting an altered expression of one or more genes or gene products or an altered activity of said one or more gene products relative to a control cell, said one or more genes or gene products being correlated to myelination and/or remyelination; and (c) selecting said agent as a candidate if the level of expression of said gene or gene product, or the level of activity of said gene product is modulated relative to said control cell.

In a related but separate embodiment, the present invention provides a method for identifying a candidate substance for promoting remyelination. The method comprises the steps of (a) providing a plurality of glial cells, at least one member of the plurality comprising a first transgene encoding a first fluorescent marker protein and a second transgene encoding a second fluorescent marker protein, wherein said second marker protein is distinguishable from said first marker protein, wherein expression of said first and said second marker protein is temporally controlled by an exogenous agent such that expression of said first marker protein occurs in myelinating glial cells existing prior to induction by said exogenous agent, and wherein expression of said second marker protein occurs in remyelinating glial cells upon induction by said exogenous agent; (b) administering said exogenous agent; (c) subjecting said cells to a demyelination insult; (d) exposing said cells to a candidate substance; and (e) detecting a fluorescent signal from said first and/or said second marker protein as compared to a control, wherein a decrease in said fluorescent signal of said second marker protein after exposure to said candidate substance indicates that said substance inhibits remyelination; and wherein an increase in said fluorescent signal indicates that said candidate substance promotes remyelination.

The present invention provides another method for testing for a biologically active agent that modulates a phenomenon associated with a demyelination disorder. The method involves the steps of: (a) administering a candidate biologically active agent to a test transgenic animal generated by method described herein; (b) inducing neuronal demyelination in said test animal, and (c) allowing said test animal to recover from the demyelination induction for a sufficient amount of time so that remyelination of a demyelinated lesion is exhibited, whereby remyelination is detected through identification of expression of one or more reporter gene products; and (d) determining the effect of said agent upon a phenomenon associated with a demyelination disorder, where a reduction or increase of expression of said one or more reporter gene products indicates that said biologically active agent modulates remyelination.

The phenomenon associated with remyelination is characterized in neuronal cells in the central nervous system. Furthermore, the phenomenon associated with remyelination is characterized by an increase in myelinated axons in the central nervous system or peripheral nervous system.

In a related but separate embodiment, the present invention provides a method for determining whether a candidate substance modulates remyelination. The method comprises the steps of (a) providing a subject transgenic animal; (b) administering said exogenous agent to induce expression of said third transgene; (c) subjecting said animal to a demyelination insult; (d) exposing said animal to said candidate substance; and (e) detecting a fluorescent signal from said first and/or said second marker protein as compared to a control, wherein a decrease in said fluorescent signal of said second marker protein after exposure to said candidate substance indicates that said substance inhibits remyelination; and wherein an increase in said fluorescent signal indicates that said candidate substance promotes remyelination.

Further provided in the present invention is a vector useful for generating the subject transgenic animals and cells thereof. The subject vector comprises (a) a first transgene encoding a first marker protein, wherein expression of said first transgene is under the control of a glial cell specific regulatory element; and (b) a second transgene encoding a second marker protein, wherein said second marker protein is expressed when expression of said first marker protein is suppressed, and wherein said first and second marker proteins are different proteins. Where desired, the vector encodes a first and a second marker protein each of which is fluorescent and emits a different detectable wavelength. The present invention also provides cells comprising the subject vectors. In one aspect, the cells are neural cells, including without limitation neurons and glial cells.

### BRIEF DESCRIPTION OF THE DRAWINGS

FIG. 1: illustrates myelination and remyelination scenarios with the present invention.

FIG. 2: illustrates the corresponding constructs for myelination and remyelination scenarios.

FIG. 3: illustrates a schematic for PDGFα/CreER T2 and PLP-TCR/mCherry-F/EGFP-F; without tamoxifen the construct expresses red label (mCherry) while with tamoxifen, Cre is induced and excises mCherry, and the green label (EGFP) is expressed.

FIG. 4: depicts a schematic of the CreER^{T2} knockin targeting construct into the mouse gene that encodes the Platelet-Derived Growth Factor Receptor-alpha (PDGFR-α) to generate the PDGFR-α/CreER^{T2} mice.

FIG. 5: depicts sequencing primers for the CreER^{T2} knockin targeting construct.

FIG. 6: depicts PCR screening strategy and primers to identify positive CreER^{T2} knockin recombinant ES clones.

FIG. 7: depicts PCR products from PCR screening strategy of FIG. 6.

FIG. 8: depicts a schematic of the mCherry-EGFP knockin targeting construct into the mouse gene that encodes PLP to generate the PLP/loxP-mCherry-loxP-EGFP mice.

FIG. 9: depicts a vector map of the mCherry-F plasmid.

FIG. 10: depicts a vector map of the EGFP-F plasmid.

FIG. 11: depicts a vector map of the pBS246 plasmid.

FIG. 12: depicts a diagram of the PLP promoter cassette.

FIG. 13 illustrates a schematic for SPRR1/Cre and Tau/mCherry/EGFP; without injury the construct expresses red label (mcherry) while with injury, Cre is induced and excises mCherry, and the green label (EGFP) is expressed.

FIG. 14: illustrates a schematic for Nestin/CreER T2 and TIMP 1/mCherry/EGFP; without tamoxifen the construct expresses red label (mcherry) while with tamoxifen, Cre is induced and excises mcherry, and the green label (EGFP) is expressed

### DETAILED DESCRIPTION OF THE EMBODIMENTS

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

General Techniques:

The practice of the present invention employs, unless otherwise indicated, conventional techniques of immunology, biochemistry, chemistry, molecular biology, microbiology, cell biology, genomics and recombinant DNA, which are within the skill of the art. See Sambrook, Fritsch and Maniatis, MOLECULAR CLONING: A LABORATORY MANUAL, 2^{nd} edition (1989); CURRENT PROTOCOLS INN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

### DEFINITIONS

As used in the specification and claims, the singular form “a”, “an” and “the” include plural references unless the context clearly dictates otherwise. For example, the term “a cell” includes a plurality of cells, including mixtures thereof.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

A “nucleotide probe” or “probe” refers to a polynucleotide used for detecting or identifying its corresponding target polynucleotide in a hybridization reaction.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR, or the enzymatic cleavage of a polynucleotide by a ribozyme.

The term “hybridized” as applied to a polynucleotide refers to the ability of the polynucleotide to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. The hybridization reaction may constitute a step in a more extensive process, such as the initiation of a PCR reaction, or the enzymatic cleavage of a polynucleotide by a ribozyme.

As used herein, “expression” refers to the process by which a polynucleotide is transcribed into mRNA and/or the process by which the transcribed mRNA (also referred to as “transcript”) is subsequently being translated into peptides, polypeptides, or proteins. The transcripts and the encoded polypeptides are collectedly referred to as “gene product.” If the polynucleotide is derived from genomic DNA, expression may include splicing of the mRNA in a eukaryotic cell.

“Differentially expressed,” as applied to nucleotide sequence or polypeptide sequence in a subject, refers to over-expression or under-expression of that sequence when compared to that detected in a control. Underexpression also encompasses absence of expression of a particular sequence as evidenced by the absence of detectable expression in a test subject when compared to a control.

The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” refers to either natural and/or unnatural or synthetic amino acids, including glycine and both the D or L optical isomers, and amino acid analogs and peptidomimetics.

As used herein, “myelinating cell” refers to those cells capable of producing myelin which insulates axons in the nervous system. Exemplary myelinating cells are oligodendrocytes responsible for producing myelin in the central nervous system, and Schwann cells responsible for producing myelin in the peripheral nervous system.

The term “remyelinating” or “remyelination” refers to regeneration of myelin, e.g., in response to a demyelination insult.

A “subject,” “individual” or “patient” is used interchangeably herein, which refers to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

The “biologically active agents” that are employed in the animal model or cell culture assays described herein may be selected from the group consisting of a biological or chemical compound such as a simple or complex organic or inorganic molecule, peptide, peptide mimetic, protein (e.g. antibody), liposome, small interfering RNA, or a polynucleotide (e.g. anti-sense). Furthermore, such agents include complex organic or inorganic molecules can include a heterogeneous mixture of compounds, such as crude or purified plant extracts.

A “promoter element” is a regulatory sequence that promotes transcription of a gene that is linked to such a sequence. The regulatory sequence can include enhancer sequences or functional portions thereof.

A “control” is an alternative subject, cell or sample used in an experiment for comparison purpose.

A “floxed” gene refers to a gene that is flanked by two lox sites and where the gene contains a transcription terminator (e.g., stop signal).

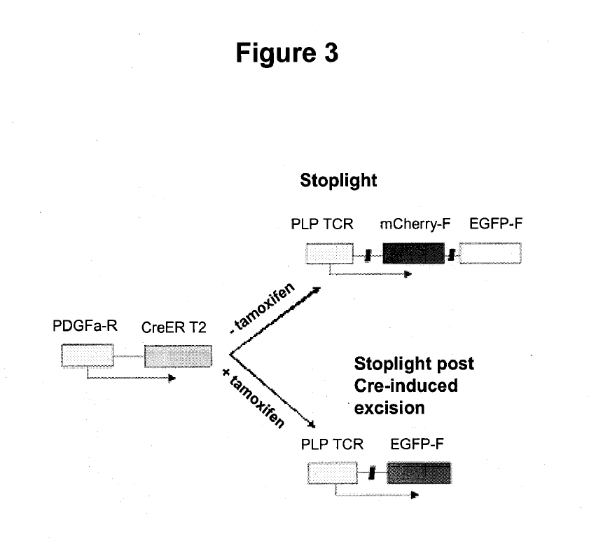

A “stoplight construct” refers to a gene construct comprising a first gene that is floxed that is further operably linked to a second gene. Therefore, if the first floxed gene is removed through recombinase (e.g., Cre) mediated recombination, the second gene would be expressed (for example, FIG. 3). The “stoplight construct” may also be referred to as “stoplight cassette”, and may optionally be operably linked to a promoter sequence.

Vectors:

One aspect of the present invention is the vectors, or targeting vectors/constructs, used for generating transgenic animals with neural cell-specific expression of the transgenes. Another aspect of the present invention is the use of the vectors in host cells, for example transfection of cells with vectors of the present invention for cell-based assays.

In one aspect, the subject vector comprises (a) a first transgene encoding a first marker protein, wherein expression of said first transgene is under the control of a glial cell specific regulatory element; and (b) a second transgene encoding a second marker protein, wherein said second marker protein is expressed when expression of said first marker protein is suppressed, and wherein said first and second marker proteins are different proteins. Where desired, the vector encodes a first and a second marker protein each of which is fluorescent and emits a different detectable wavelength.

In a preferred embodiment, the targeting vectors or constructs are used to generate a transgenic animal having a transgenic nucleotide sequence operably linked to a neural cell-specific promoter sequence, where said nucleotide sequence encodes a recombinase. In a preferred embodiment, the first targeting vector or construct is designed such that expression of the cognate recombinase protein that exploits the transcriptional control region from a gene sequence that operates in a cell-specific manner. In preferred embodiments, the recombinase is engineered to be active only when induced, for example, by an exogenous agent.

In another aspect, a second targeting vector or construct is designed for generating a second transgenic animal that expresses a reporter gene/product that exploits the transcriptional control region from a gene sequence that operates in a cell-specific manner. In one embodiment, the gene construct to be introduced comprises two genes of interest, in combination with a promoter/enhancer element form one transcriptional unit. In preferred embodiments, the first gene of interest comprises a transcription termination signal (e.g., stop codon) and is flanked by sequences capable of recognition by a cognate recombinase protein (e.g the recombinase encoded by the first targeting construct). Furthermore, operably linked to this gene is the second gene of interest that is only expressed if the first gene of interest is excised, where such excision can be mediated in the presence of a recombinase protein recognizing the cognate flanking sequences. In a further embodiment, the first and second transgenes are present in a common animal (for example, the animals described are mated and progeny expressing both transgenes are selected) or a host cell (for example, a host cell comprising both vectors, or transgenes), wherein inducible expression of the recombinase permits differential expression of the marker proteins in specific subpopulations of glial cells. In preferred embodiments, the expression of the marker proteins in different subpopulations allows differentiation between developmental myelinating cells and remyelinating cells.

In preferred embodiments, the first targeting construct or vector used to generate a first transgenic animal having stably integrated into its genome encodes a recombinase, wherein the recombinase is a Cre recombinase, which recognizes the cognate recognition sequences, loxP sequences (i.e., loxP sites). Recognition sequences are known in the art, and represent particular DNA sequences which a protein, DNA, or RNA molecule (e.g., restriction endonuclease, a modification methylase, or a recombinase) recognizes and binds. For example, the recognition sequence for Cre recombinase is loxP which is a 34 base pair sequence comprised of two 13 base pair inverted repeats (serving as the recombinase binding sites) flanking an 8 base pair core sequence. (See Sauer, Curr. Opin. Biotech. 5:521-527 (1994)). Other examples of recognition sequences are the attB, attP, attL, and attR sequences which are recognized by the recombinase enzyme λ Integrase. attB is an approximately 25 base pair sequence containing two 9 base pair core-type Int binding sites and a 7 base pair overlap region. attP is an approximately 240 base pair sequence containing core-type Int binding sites and arm-type Int binding sites as well as sites for auxiliary proteins IHF, FIS, and Xis. See Landy, Curr. Opin. Biotech. 3:699-707 (1993). Such sites can also be engineered according to the present invention to enhance recombination utilizing methods and products as known in the art such as disclosed in the disclosure by Hartley et al., U.S. Patent Application Publication No. 20060035269.

The Cre recombinase may be wild type or a variant of the wild type. In preferred embodiments, the Cre recombinase is inducible in the transgenic animal (or transgenic cells). Variant Cre recombinases have broadened specificity for the site of recombination. Specifically, the variants mediate recombination between sequences other than the loxP sequence and other lox site sequences on which wild type Cre recombinase is active. In general, the disclosed Cre variants mediate efficient recombination between lox sites that wild type Cre can act on (referred to as wild type lox sites), between variant lox sites not efficiently utilized by wild type Cre (referred to as variant lox sites), and between a wild type lox site and a variant lox site. For example, the Cre variants can be used in any method or technique where Cre recombinase (or other, similar recombinases such as FLP) can be used. In addition, the Cre variants allow different alternative recombinations to be performed since the Cre variants allow much more efficient recombination between wild type lox sites and variant lox sites. Control of such alternative recombination can be used to accomplish more sophisticated sequential recombinations to achieve results not possible with wild type Cre recombinase. Variant Cre recombinases are known in the art, such as disclosed in the disclosure of U.S. Pat. No. 6,890,726. The inducibility of Cre activity may be controlled by the localization of the Cre protein. For example, the Cre protein may be a fusion of the Cre recombinase with a mutated version of the estrogen receptor, resulting in the Cre fusion, CreER^{t2}. In the absence of ligand, CreER^{t2} is cytoplasmic. However, following administration of a synthetic steroid hormone (tamoxifen), the Cre ER^{t2} protein translocates into the nucleus where it is functional (i.e., tamoxifen-inducible).

In another aspect of the invention, the second targeting vector comprises a nucleotide sequence operably linked to a neural cell-specific promoter sequence, where said nucleotide sequence comprises two or more genes encoding marker proteins. In one embodiment, the two or more genes each encode a fluorophore or a fluorescent protein. In another embodiment, the transgenic nucleotide sequence comprises a first and second gene in tandem and operably linked to a neural cell-specific promoter, where the first gene encodes a terminator or stop codon thus if the first gene is expressed the second gene is not. In yet a further embodiment, the first gene is also flanked by recognition sequences that provide a means for the first gene, including the termination/stop sequence, to be removed. Such a removal or excision can be enzymatic, e.g., via a recombinase protein that recognizes said recognition sequences.

In one aspect, the second targeting vector comprises a stoplight cassette (e.g. FIG. 3), wherein the stoplight cassette is under the control of a promoter/enhancer element or regulatory sequence. The stoplight cassette comprises a first and second gene, each encoding a different fluorescent protein, where the first gene contains a termination signal and is further flanked by recognition sequences for a recombinase enzyme. Therefore, if the first gene is expressed the second gene is precluded from expression. Non-exclusive examples of marker genes that can be used in the present invention include reef coral fluorescent proteins (RCFPs), HcRed1, AmCyan1, AsRed2, mRFP1, DsRed1, jellyfish fluorescent protein (FP) variants, red fluorescent protein, green fluorescent protein (GFP), blue fluorescent protein, luciferase, GFP mutant H9, GFP H9-40, EGFP, tetramethylrhodamine, Lissamine, Texas Red, EBFP, ECFP, EYFP, Citrine, Kaede, Azami Green, Midori Cyan, Kusabira Orange and naphthofluorescein, or enhanced functional variants thereof. Many genes encoding fluorophore proteins markers are known in the art, which markers are capable of use in the present invention. See, website: <cgr.harvard.edu/thomlab/gfps.htm>. Mutated version of fluorescence proteins that emit light of greater intensity or which exhibit wavelength shifts can also be utilized in the compositions and methods of the present invention; such variants are known in the art and commercially available. (See Clontech Catalogue, 2005). In yet another embodiment, each of the fluorescent labels is farnesylated so that the fluorescent labels will be membrane associated. In preferred embodiments, the stoplight cassette encodes a red fluorescent label as first marker/label (e.g., mcherry flanked by loxP sites) and EGFP as the second fluorescent label (FIG. 3 Stop Light). See, e.g., Yang and Hughes, BioTechniques, 31:1036-41 (2001) (teaching Red/Green reporter of Cre Expression in HEK 293 cells).

The first and second targeting constructs preferably express their transgenes under the control of a promoter or regulatory sequence, in particular those available for expressing transgenes in the central nervous systems. The regulatory sequences may allow ectopic expression of transgenes in the central nervous system in particular neural cells, specifically in the oligodendrocytes, Schwann cells, astrocytes or Muller cells. Examples of neural cell-specific promoters are known in the art, such as disclosed in U.S. Patent Application Publication No. 2003/0110524; See also, the website <chinook.uoregon.edu/promoters.html>. Exemplary transcriptional regulatory sequences include transcriptional regulatory sequences selected from the genes encoding the following proteins: the PDGFα receptor, proteolipid protein (PLP), the glial fibrillary acidic gene (GFAP), myelin basic protein (MBP), neuron specific enolase (NSE), oligodendrocyte specific protein (OSP), myelin oligodendrocyte glycoprotein (MOG) and microtubule-associated protein 1B (MAP1B), Thy1.2, CC1, ceramide galactosyltransferase (CGT), myelin associated glycoprotein (MAG), oligodendrocyte-myelin glycoprotein (OMG), cyclic nucleotide phosphodiesterase (CNP), NOGO, myelin protein zero (MPZ), peripheral myelin protein 22 (PMP22), protein 2 (P2), tyrosine hydroxylase, BSF1, dopamine 3-hydroxylase, Serotonin 2 receptor, choline acetyltransferase, galactocerebroside (GalC), and sulfatide.

In some embodiments, the regulatory sequence can be altered or modified to enhance expression (i.e., increase promoter strength). For example, intronic sequences comprising enhancer function can be utilized to increase promoter function. The myelin proteolipid protein (PLP) gene comprises an intronic sequence that functions as an enhancer element. This regulatory element/region ASE (for antisilencer/enhancer) is situated approximately 1 kb downstream of exon 1 DNA and encompasses nearly 100 bp. See, Meng et al., J. Neurosci. Res. 82:346-356 (2005).

Furthermore, where expression of the transgene in a particular subcellular location is desired, the transgene can be operably linked to the corresponding subcellular localization sequences by recombinant DNA techniques widely practiced in the art. Exemplary subcellular localization sequences include but are not limited to (a) a signal sequence that directs secretion of the gene product outside of the cell; (b) a membrane anchorage domain that allows attachment of the protein to the plasma membrane or other membraneous compartment of the cell; (c) a nuclear localization sequence that mediates the translocation of the encoded protein to the nucleus; (d) an endoplasmic reticulum retention sequence (e.g. KDEL sequence) that confines the encoded protein primarily to the ER; (e) proteins can be designed to be farnesylated so as to associate the protein with cell membranes; or (f) any other sequences that play a role in differential subcellular distribution of a encoded protein product.

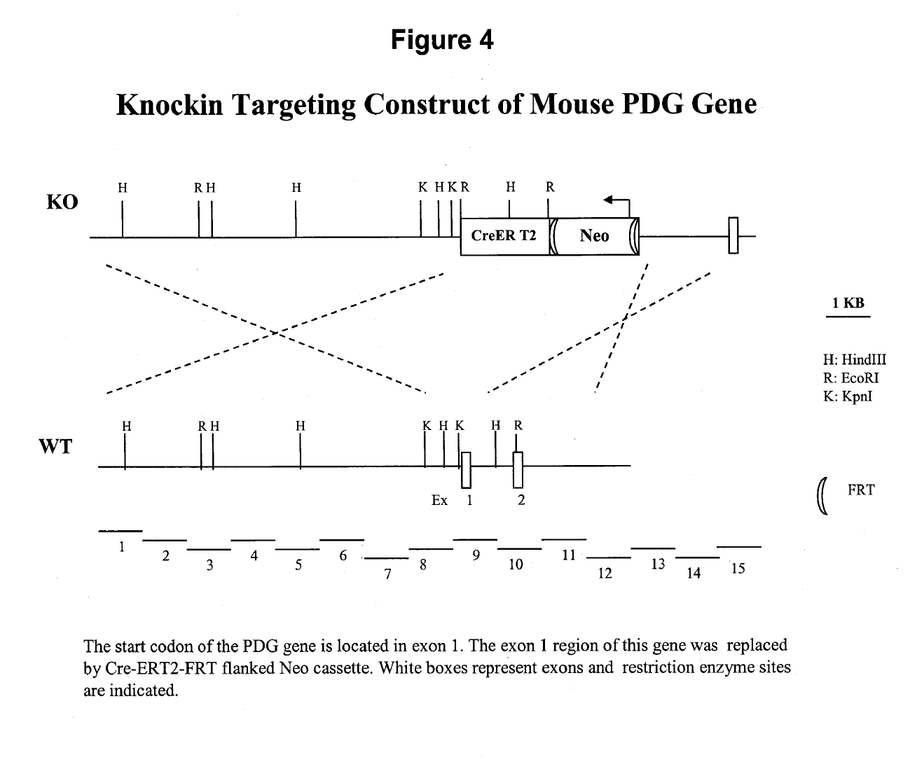

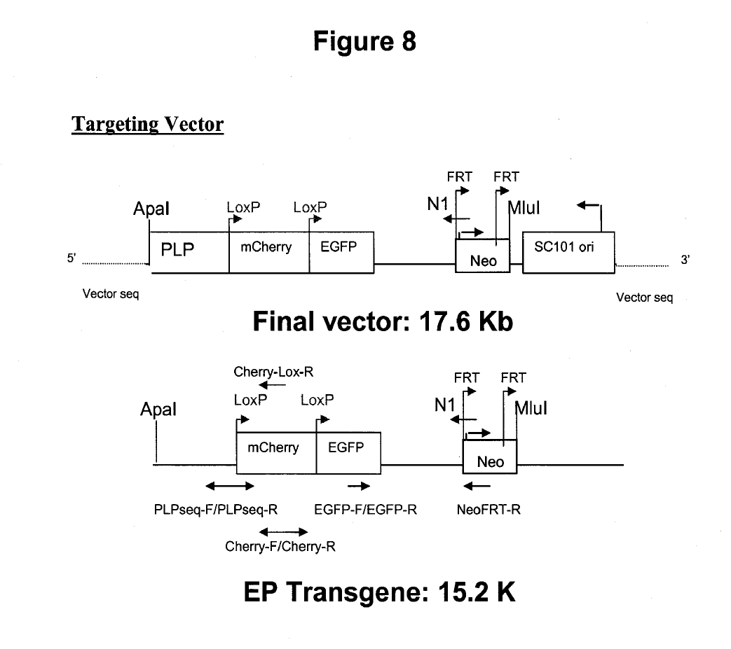

In a preferred embodiment, the first vector comprises a promoter captured from the endogenous PDGFα receptor gene, wherein PDGFα is expressed in oligodendrocyte progenitor cells, but not in mature oligodendrocytes. The PDGFα is operably linked to the sequence encoding CreER^{T2} in a vector (FIG. 4) and may be used to generate a transgenic animal, wherein the animal is preferably a mouse. The resulting transgenic mouse is PDGFα/CreER^{T2}. In a further embodiment, the second targeting construct comprises a regulatory sequence of PLP, a promoter sequence specific for myelinating oligodendrocytes, and the marker genes are encoded with subcellular localization sequences, in particular the marker proteins are designed to be farnesylated. In preferred embodiments, the vector comprises a PLP promoter controlling the expression of mcherry and EGFP, both of which are farnesylated when expressed. A targeting construct to generate a PLP/loxP-mCherry-loxP-EGFP mouse is depicted in FIG. 8.

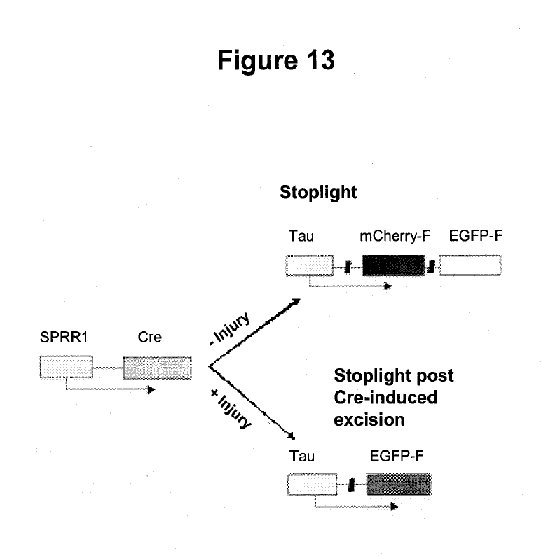

In yet another embodiment, the first vector comprises a promoter derived from a gene preferentially expressed in neurons after axotomy as compared to during neuronal development. One example is SPRR1 gene (Wang et al., Disease Gene Candidates Revealed by Expression Profiling of Retinal Ganglion Cell Development, J. Neurosci. 27 in press (2007)). In an illustrative schematic shown in FIG. 13, the SPRR1 promoter is operably linked to the sequence encoding Cre in a vector and can be used to generate a transgenic animal, wherein the animal is preferably a mouse. The resulting transgenic mouse is SPRR1/Cre. The second targeting construct comprises a regulatory sequence, wherein the regulatory sequence is a promoter of Tau. Tau is an abundant protein in neurons, and in other embodiments, regulatory sequences from other proteins expressed abundantly in neurons may be used. The Tau promoter is operably linked to the sequence encoding mcherry and EGFP, resulting in a transgenic mouse Tau/loxP-mCherry-loxP-EGFP. A double transgenic mouse may be generated from the SPRR1/Cre and Tau/loxP-mCherry-loxP-EGFP mice, and neurons prior to injury may fluoresce red (mCherry), whereas regenerated neurons after axotomy should fluoresce green (EGFP) (FIG. 13).

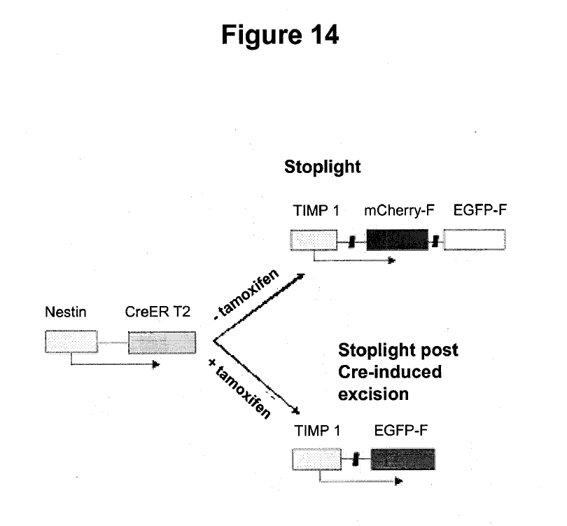

In another embodiment, the first vector comprises a promoter derived from a cellular gene expressed in neuronal precursor cells. One example is the endogenous Nestin gene. In an illustrative schematic shown in FIG. 14, the Nestin promoter is operably linked to the sequence encoding inducible CreER^{T2} in a vector and can be used to generate a transgenic animal, wherein the animal is preferably a mouse. The resulting transgenic mouse is Nestin/CreER^{T2}. The second targeting construct comprises a regulatory sequence, wherein the regulatory sequence is a promoter of TIMP 1. TIMP 1 expression increases during neuronal development and neuronal regeneration (Wang et al., Disease Gene Candidates Revealed by Expression Profiling of Retinal Ganglion Cell Development, J. Neurosci. 27 in press (2007)). In other embodiments, regulatory sequences of other genes with increased expression during neuronal development and neuronal regeneration may be used. The TIMP 1 promoter is operably linked to the sequence encoding mcherry and EGFP, resulting in a transgenic mouse TIMP 1/loxP-mCherry-loxP-EGFP. A double transgenic mouse may be generated from the Nestin/CreER^{T2} and TIMP 1/loxP-mCherry-loxP-EGFP mice, and neurons fluoresce red (mcherry) when not induced with tamoxifen, and fluoresce green (EGFP) when treated with taxmofen. (FIG. 14).

A vast number of genetic vehicles suitable for the present invention are available in the art. They include both viral and non-viral expression vectors. Non-limiting exemplary viral expression vectors are vectors derived from RNA viruses such as retroviruses, and DNA viruses such as adenoviruses and adeno-associated viruses. Non-viral expression vectors include but are not limited to plasmids, cosmids, and DNA/liposome complexes. The genetic vehicles can be engineered to carry regulatory sequences that direct tissue specific, cell specific, or even organelle specific expression of the exogenous genes carried therein.

Vectors that can be utilized with one or more composition or methods of the present invention include derivatives of SV-40, adenovirus, retrovirus-derived DNA sequences and shuttle vectors derived from combinations of functional mammalian vectors and functional plasmids and phage DNA. Eukaryotic expression vectors are well known, e.g. such as those described by Southern and Berg, J. Mol. Appl. Genet. 1:327-341 (1982); Subramini et al., Mol. Cell. Biol. 1:854-864 (1981), Kaufmann and Sharp, J. Mol. Biol. 159:601-621 (1982); Scahill et al., Proc. Natl. Acad. Sci. USA 80:4654-4659 (1983) and Urlaub and Chasin, Proc. Natl. Acad. Sci. USA 77:4216-4220 (1980), which are hereby incorporated by reference. The vector used in the methods of the present invention may be a viral vector, preferably a retroviral vector. Replication deficient adenoviruses are preferred. For example, a “single gene vector” in which the structural genes of a retrovirus are replaced by a single gene of interest, under the control of the viral regulatory sequences contained in the long terminal repeat, may be used, e.g., Moloney murine leukemia virus (MoMulV), the Harvey murine sarcoma virus (HaMuSV), murine mammary tumor virus (MuMTV) and the murine myeloproliferative sarcoma virus (MuMPSV), and avian retroviruses such as reticuloendotheliosis virus (Rev) and Rous Sarcoma Virus (RSV), as described by Eglitis and Andersen, BioTechniques 6:608-614 (1988), which is hereby incorporated by reference.

If desired, the genetic vehicles can be inserted into a host cell (e.g., myelinating cells such as oligodendrocytes or Schwann cells) by any methods known in the art. Suitable methods may include transfection using calcium phosphate precipitation, DEAE-dextran, electroporation, or microinjection.

Transgenic Animals:

One aspect of the present invention comprises compositions and methods utilizing a double transgenic animal that provides a time-controlled and cell-specific expression of marker genes/products thus allowing identification of newly formed myelin or remyelination, as compared to developmental or pre-existing myelin.

In one embodiment, the subject transgenic animal comprises: a first transgene encoding a first fluorescent marker protein and a second transgene encoding a second fluorescent marker protein, wherein said second marker protein is distinguishable from said first marker protein, and wherein expression of said first and said second marker protein is temporally controlled by an exogenous agent, and said expression occurs in a subpopulation of glial cells. In one aspect, the subpopulation of glial cells are mature oligodendrocytes. In another aspect, the subpopulation of glial cells are remyelinating oligodendrocytes. In yet another aspect, the exogenous agent induces expression of a third transgene in said subpopulation of glial cells so as to temporally control expression of said first and said second marker protein. In some instances, expression of said first marker protein occurs in myelinating glial cells existing prior to induction by said exogenous agent, and wherein expression of said second fluorescent marker protein occurs in remyelinating glial cells upon induction by said exogenous agent.

In another embodiment, the transgenic animals contain: 1) a neural specific promoter expressed in a specific subpopulation of neural cells with an inducible recombinase under the control of the promoter and 2) a stoplight cassette of two marker genes under the expression of another neural specific promoter of another subpopulation of neural cells. In preferred embodiments, the first promoter is expressed specifically in progenitor cells and the second promoter expressed specifically in mature cells.

Another aspect of the invention is the stoplight construct comprising a marker gene/product whose expression is remyelination-specific, so that detection/quantification of expression correlates with remyelination. In yet a further aspect, increase or decrease in such expression correlates with increased or decreased remyelination. The vectors described above may be used to generate transgenic animals of the present invention.

In a preferred embodiment, the transgenic animal, preferably a mouse, comprises the transgenes CreER^{T2} and loxP-mCherry-loxP-EGFP, wherein CreER^{T2} is under the control of the endogenous PDGFα promoter and loxP-mCherry-loxP-EGFP is under the control of the PLP promoter. The transgenic mouse may be generated by crossing PDGFα/CreER^{T2} mice with PLP/loxP-mCherry-loxP-EGFP mice. The PDGFα/CreER^{T2} mice and PLP/loxP-mCherry-loxP-EGFP mice may be generated by using the targeting strategies and vectors as depicted in FIGS. 4 and 8.

In yet another embodiment, the transgenic animal, preferably a mouse, comprises the transgenes Cre and loxP-mCherry-loxP-EGFP, wherein Cre is under the control of the SPRR1 promoter and loxP-mCherry-loxP-EGFP is under the control of the Tau promoter. The transgenic mouse may be generated by crossing SPRR1/Cre mice and Tau/loxP-mCherry-loxP-EGFP mice. In another embodiment, the transgenic animal, preferably a mouse, comprises the CreER^{T2} transgenes under the control of the Nestin promoter and loxP-mCherry-loxP-EGFP under the control of the TIMP 1 promoter. The transgenic mouse may be generated by crossing Nestin/CreER^{T2} and TIMP 1/loxP-mCherry-loxP-EGFP mice.

The transgenic animals are designed utilizing gene targeting techniques known in the art. Gene targeting represents the directed modification of a chromosome locus by homologous recombination with an exogenous DNA sequence homologous with the targeted endogenous sequence. A distinction is made between different types of gene targeting. Thus, gene targeting may be used to modify, and usually increase, the expression of one or several endogenous genes, or to replace an endogenous gene by an exogenous gene, or to place an exogenous gene under the control of elements regulating the gene expression of the particular endogenous gene that remains active. In this case, gene targeting is called “Knock-in” (KI). Alternatively, gene targeting may be used to reduce or eliminate the expression of one or several genes, and this type of gene targeting is called “Knock-out” (KO) (See, e.g., Bolkey et al., Ann. Rev. Genet. 23:199-225 (1989)).

Methods of generating transgenic cells according to the invention are well known to those skilled in the art. Various techniques for transfecting mammal cells have been described (Gordon., Intl. Rev. Cytol 115:171-229 (1989)). The transgene according to the invention, optionally included in a linearized or non-linearized vector or in the form of a vector fragment, may be introduced into the host cell by standard methods, for example such as micro-injection into the nucleus (U.S. Pat. No. 4,873,191), transfection by precipitation with calcium phosphate, lipofection, electroporation (Lo, Mol. Cell. Biol. 3:1803-1814 (1983)), thermal shock, transformation with cationic polymers (PEG, polybrene, DEAE-Dextran, etc.), viral infection (Van der Putten et al., Proc. Natl. Acad. Sci. USA 82, 6148-6152 (1985)), or sperm (Lavitrano et al, Cell 57:717-723 (1989)).

A transgenic animal is engineered by insertion of a genetic construct into the pronucleus (preferably the male pronucleus) of a mammalian zygote, and allowing stable genomic integration to occur naturally. The zygote is then transferred to a receptive uterus, and allowed to develop to term. While the mouse is a preferred species, rats and rabbits are also potential candidates for pronuclear insertion. The genetic construct which renders the zygote transgenic comprises a gene construct that targets an endogenous gene to be exploited (e.g., PDGFα receptor gene), which gene can be mutated and/or further modified to comprise desired elements (e.g., a exogenous promoter/enhancer element and/or a gene of interest).

Advances in technologies for embryo micromanipulation now permit introduction of heterologous DNA into fertilized mammalian ova as well. For instance, totipotent or pluripotent stem cells can be transformed by microinjection, calcium phosphate mediated precipitation, liposome fusion, retroviral infection or other means. The transformed cells are then introduced into the embryo, and the embryo will then develop into a transgenic animal. In a preferred embodiment, developing embryos are infected with a viral vector containing a desired transgene so that the transgenic animals expressing the transgene can be produced from the infected embryo. In another preferred embodiment, a desired transgene is coinjected into the pronucleus or cytoplasm of the embryo, preferably at the single cell stage, and the embryo is allowed to develop into a mature transgenic animal. These and other variant methods for generating transgenic animals are well established in the art and hence are not detailed herein. See, for example, U.S. Pat. Nos. 5,175,385 and 5,175,384.

In one or more aspects of the invention disclosed herein, a desired transgene may be integrated as a single copy or in concatamers, e.g., head-to-head tandems or head-to-tail tandems. The desired transgene may also be selectively introduced into and activated in a particular tissue or cell type, preferably cells within the central nervous system. The regulatory sequences required for such a cell-type specific activation will depend upon the particular cell type of interest, and will be apparent to those of skill in the art. Preferably, the targeted cell types are located in the nervous systems, including the central and peripheral nervous systems.

As noted above, transgenic animals can be broadly categorized into two types: “knockouts” and “knockins”. A “knockout” has an alteration in the target gene via the introduction of transgenic sequences that result in a decrease of function of the target gene, preferably such that target gene expression is insignificant or undetectable. A “knockin” is a transgenic animal having an alteration in a host cell genome that results in an augmented expression of a target gene, e.g., by introduction of an additional copy of the target gene, or by operatively inserting a regulatory sequence that provides for enhanced expression of an endogenous copy of the target gene. The knock-in or knock-out transgenic animals can be heterozygous or homozygous with respect to the target genes. Both knockouts and knockins can be “bigenic”. Bigenic animals have at least two host cell genes being altered. A preferred bigenic animal carries a transgene encoding a neural cell-specific recombinase and another transgenic sequence that encodes neural cell-specific marker genes. The transgenic animals of the present invention can broadly be classified as Knockins.

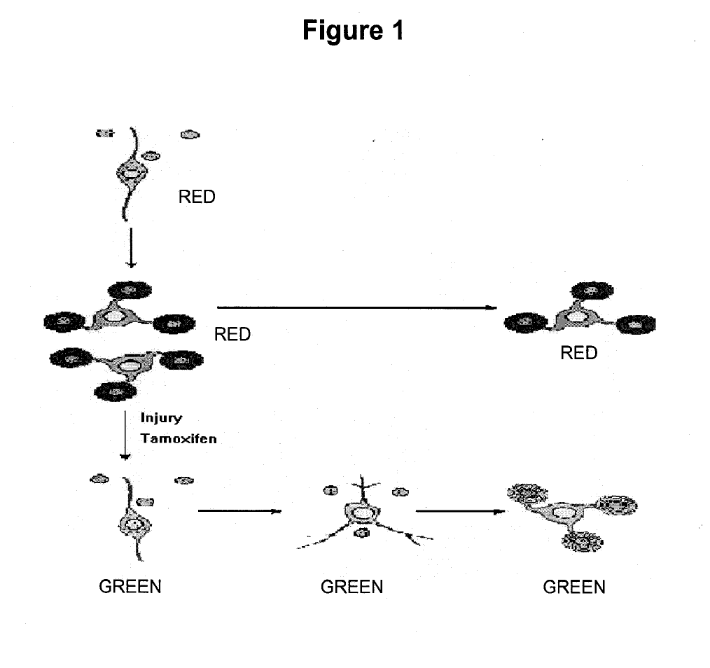

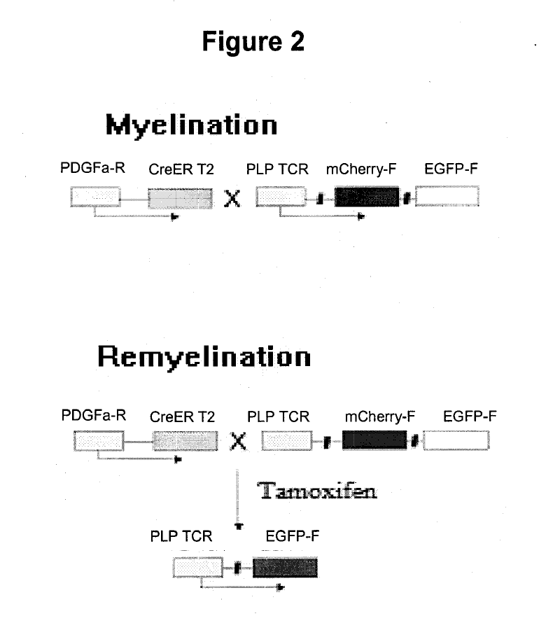

In the present invention, the transgenic animals are designed to provide a model system for identifying and quantifying remyelination. Such quantification can occur at any time during the animal's life span, including before or after post demyelination insult. The transgenic model system can also be used for the development of biologically active agents that promote or are beneficial for neuronal remyelination. Furthermore, the model system can be utilized to assay whether a test agent impart a detrimental effect or reduces remyelination, e.g., post demyelination insult (FIGS. 1 and 2). Moreover, neural cells can be isolated from the transgenic animals of the invention for further study or assays conducted in a cell-based or cell culture setting, including ex vivo techniques.

The animal models of the present invention encompass any non-human vertebrates that are amenable to procedures yielding a neuronal demyelination condition in the animal's nervous systems including the central and peripheral nervous system. Preferred model organisms include but are not limited to mammals, primates, and rodents. Non-limiting examples of the preferred models are rats, mice, guinea pigs, cats, dogs, rabbits, pigs, chimpanzees, and monkeys. The test animals can be wildtype or transgenic. In one embodiment, the animal is a rodent. In yet another embodiment, the animal is a mouse. In another embodiment, the animal is from a simian species. In yet another embodiment, the animal is a marmoset monkey, which monkeys are utilized in examining neurological disease (e.g., Eslamboi, Brain Res. Bull. 68:140-149 (2005); Kirik et al., Proc. Natl. Acad. Sci. 100:2884-2889 (2004)).

Accordingly, the present invention provides a method of using animal models for detecting and quantifying remyelination in a cell-specific manner. In such an embodiment, the method comprises the steps of: (a) inducing demyelination insult in the transgenic animal of the invention; (b) allowing time for myelin repair occur; (c) detecting and/or quantifying expression of cell-specific marker gene(s); (d) determining if and how much remyelination has occurred.

In another aspect of the invention, the present invention provides a method of testing a biologically active agent for remyelination modulation activity. The method comprises the steps of: (a) inducing demyelination insult in the transgenic animal of the invention; (b) allowing time for myelin repair to occur; (c) administering a test agent to the animal; (d) detecting and/or quantifying expression of cell-specific marker gene(s) before and after step (c); (e) detecting if and how much remyelination has occurred in step (d); (f) determining the test agent to have remyelination modulation activity if expression of remyelination-specific marker proteins is up- or down-regulated in response to administration of the test agent. In some embodiments, detection is made at various time points and administration of a test agent can be repeated during the course of the assay, as well as using different dosing regimens.

In yet another aspect of the invention, the present invention provides a method of testing a candidate agent, for remyelination inducing or promoter activity. The method comprises the steps of: (a) inducing demyelination insult in the transgenic animal of the invention; (b) allowing time for myelin repair to occur; (c) administering a test agent to the animal; (d) detecting and/or quantifying expression of cell-specific marker gene(s) before and after step (c); (e) detecting if and how much remyelination has occurred in step (d); (f) determining the test agent to have remyelination inducing or promoter activity if expression of cell-specific marker proteins is increased in response to administration of the test agent. In some embodiments, the expression of the cell-specific marker protein in the test animal can be compared to a control or reference animal. In other embodiments, the expression of the cell-specific marker protein in the test animal is compared to measurements made at various time points in the same animal, where an earlier time point can be used as a reference or control time point. In yet other embodiments, the expression of the cell-specific marker protein is measured in a number of test animals, wherein measurements may be taken at various time points of the test animals, and compared to corresponding control animals. The cell-specific marker is preferably a remyelination-specific marker.

In a further aspect of the invention, the present invention provides a method of testing a candidate agent, for remyelination inhibiting or reducing activity. The method comprises the steps of: (a) inducing demyelination insult in the transgenic animal of the invention; (b) allowing time for myelin repair occur; (c) detecting and/or quantifying expression of cell-specific marker gene(s); (d) detecting and quantifying remyelination; (e) administering a candidate agent to the animal; (f) detecting and quantifying if and how much remyelination has occurred before and after step (e); and (g) determining the test agent to have remyelination inhibiting or reducing activity if expression of cell-specific marker proteins is decreased in response to administration of the test agent. In some embodiments, the expression of the cell-specific marker protein in the test animal can be compared to a control or reference animal. In other embodiments, the expression of the cell-specific marker protein in the test animal is compared to measurements made at various time points in the same animal, where an earlier time point can be used as a reference or control time point. In other embodiments, the expression of the cell-specific marker protein is measured in a number of test animals, wherein measurements may be taken at various time points of the test animals, and compared to corresponding control animals. In yet other embodiments, the expression of the cell-specific marker, preferably remyelination-specific marker proteins, is measured in the test animal and a control or reference animal, in determining whether a candidate agent has remyelination inhibiting or reducing activity. Such an agent can be categorized as a remyelination inhibitor or remyelination toxin.

In one aspect of the invention, a transgenic animal is generated having stably integrated into the genome a transgenic nucleotide sequence encoding a neural cell-specific gene operably linked to a gene encoding a protein of interest, where the cell-specific gene is expressed in a cell-specific manner concomitantly with the protein of interest, and where said genes are under control of an inducible promoter. In some embodiments the cell-specificity is to neural cells, preferably to glial cells, more preferably to astrocytes, oligodendrocytes, Schwann cells or Muller cells. In another embodiment, the gene encoding a protein of interest encodes a recombinase protein.

In yet another aspect of the invention, the subject methods involve a double transgenic (F1) animal, wherein the transgenic animal represents progeny of the single transgenic animals described herein above. The F1 animal exhibits the phenotypic characteristic of inducible expression of a cell-specific recombinase, and cell-specific expression of a first or second gene encoding fluorescent labels, where the cell-specific differential expression correlates to, thus allows for detection and quantification of, remyelination. Therefore, the (F1) animal exhibits a time controlled and cell-specific expression of fluorescent labels by oligodendrocytes in a differential fashion. In a preferred embodiment, the transgenic animal is a mouse.

Therefore, in one embodiment of the invention, tamoxifen is administered to the (F1) animal thus inducing expression of Cre recombinase (e.g., CreER^{T2} gene), allowing for Cre-dependent site-specific recombination of the “floxed” transgenic nucleotide sequence, preferably the encoding the first marker protein or label. In a preferred embodiment the CreER^{T2} gene is under control of the PDGFα receptor gene thus expressed in a cell-specific manner in oligodendrocyte progenitor cells. As a result of a Cre-dependent recombination event, the mCherry encoding sequence is excised thus allowing EGFP expression from the second transgenic nucleotide sequence. Remyelination can be detected in oligodendrocytes derived from the aforementioned progenitor cells. Thus newly formed myelin (i.e., remyelination) will fluoresce green (i.e., EGFP), while pre-existing or developmental myelin will fluoresce red (i.e. mcherry). Furthermore, since Cre expression is inducible, EGFP expression can be effected in a time-controlled manner, as well as a cell-specific manner to detect remyelination-specific myelin growth. In a preferred embodiment, the double transgenic animal is a mouse and the dual transgenes are PDGFα-receptor/CreER^{T2} and PLP/loxP-mCherry-loxP-EGFP, respectively.

Therefore, the transgenic animals of the invention provide compositions and methods that can be utilized to generate a model system for assaying remyelination. Such a model system will provide insights into elucidating mechanisms of remyelination, as well as development of therapeutic strategies for promoting remyelination. Furthermore, the expression of remyelination-specific marker proteins is easily detected and quantified utilizing techniques known in the art.

Induction of Demyelination and Evaluation of Remyelination:

The present invention provides a model system in elucidating mechanisms of remyelination, as well as development of therapeutic strategies for promoting remyelination. In preferred embodiments, the transgenic animals are double transgenic PDGFα receptor/CreER^{T2} PLP/loxP-mCherry-loxP-EGFP mice, for example, produced from mating PDGFα receptor/CreER^{T2} mice with PLP/loxP-mCherry-loxP-EGFP (Lin, et al. (2004) J. Neurosci. 24: 10074-10083; teaching methods known in the art for producing a double transgenic animal).

This double transgenic mouse is capable of temporal expression of CreER^{T2}, such as in progenitor oligodendrocytes but not mature oligondendrocytes, based on utilization of the PDGF-α receptor gene. The marker genes are under the control of the PLP promoter and are expressed in myelinating oligodendrocytes. Without induction of Cre recombinase, the mouse expresses the first marker protein (e.g. mcherry) in myelinating oligodendrocytes. The mouse may be treated with tamoxifen which induces Cre activity. Cre activity may result in expression of the second marker protein (e.g., EGFP).

Using this system, developmental myelinating and remyelinating oligodendrocytes may be distinguished by treating the mouse with tamoxifen prior to demyelination insult, and then detecting expression of the first and second marker proteins. As discussed above, tamoxifen treatment induces Cre recombinase to recognize and act upon the Lox recognition sites flanking the first of two genes encoding a distinct fluorescent marker protein (e.g. mcherry). The first marker gene comprises a termination or stop signal, thus when said first gene undergoes recombination via Cre, both the gene and the stop signal are excised, whereby the resulting transgene now comprises a PLP promoter element operably linked and effecting expression of the second fluorescent marker (e.g. EGFP). After the demyelination insult, oligodendrocyte progenitor cells containing the Cre construct will enable expression of the second fluorescent marker. As a result, in these animals, developmental myelin fluoresce via the first marker protein (e.g., red, if mcherry is the first marker gene) and the “remyelin” should fluoresce via the second fluorescent protein (e.g., green, if EGFP is the second marker gene). Alternatively, at any time point post demyelination insult, mice can be administered tamoxifen, which activates the CreER^{T2} recombinase.

A number of methods for inducing demyelination in a test animal have been established. For instance, neuronal demyelination may be inflicted by pathogens or physical injuries, agents that induce inflammation and/or autoimmune responses in the test animal. A preferred method employs demyelination-induced agents including but not limited to IFN-γ cuprizone (bis-cyclohexanone oxaldihydrazone), lysolecithin or ethidium bromide. The cuprizone-induced demyelination model is described in Matsushima et al., Brain Pathol. 11: 107-116 (2001). In this method, the test animals are typically fed with a diet containing cuprizone for a few weeks ranging from about 1 to about 10 weeks. One of ordinary skill in the art will recognize that at this point, any method known in the art for inducing demyelination in the peripheral or central nervous system can be substituted as a means for inducing demyelinating insult.

A demyelination condition in the test animal generally refers to a decrease in myelinated axons in the nervous systems (e.g., the central or peripheral nervous system), or by a reduction in the levels of markers of myelinating cells, such as oligodendrocytes and Schwann cells. If desired, demyelination can be characterized by methods known in the art. Morphologically, neuronal demyelination can be characterized by a loss of oligodendrocytes in the central nervous system or Schwann cells in the peripheral nervous system. It can also be determined by a decrease in myeliriated axons in the nervous system, or by a reduction in the levels of oligodendrocyte or Schwann cell markers. Exemplary marker proteins of oligodendrocytes or Schwann cells include, but are not limited to, CC1, myelin basic protein (MBP), ceramide galactosyltransferase (CGT), myelin associated glycoprotein (MAG), myelin oligodendrocyte glycoprotein (MOG), oligodendrocyte-myelin glycoprotein (OMG), cyclic nucleotide phosphodiesterase (CNP), NOGO, myelin protein zero (MPZ), peripheral myelin protein 22 (PMP22), protein 2 (P2), galactocerebroside (GalC), sulfatide and proteolipid protein (PLP). As such, the candidate agents identified by the subject method encompass substances that can inhibit the deleterious morphological characteristics of neuronal demyelination.

After induction of a demyelination condition by an appropriate method, the animal is allowed to recover for a sufficient amount of time to allow remyelination at or near the previously demyelinated lesions. While the amount of time required for developing remyelinated axons varies among different animals, it generally requires at least about 1 week, more often requires at least about 2 to 10 weeks, and even more often requires about 4 to about 10 weeks.

Remeylination can be ascertained by observing an increase in the cell-specific expression of a marker gene/gene product (e.g., in the central or peripheral nervous system), such as by expression of the second marker protein (e.g. EGFP) as described above. In one or more methods herein, where demyelination or myelination is sought to be identified, various markers are available in the art. Exemplary markers for identifying myelinating cells include, but are not limited to, CC1, myelin basic protein (MBP), ceramide galactosyltransferase (CGT), myelin associated glycoprotein (MAG), myelin oligodendrocyte glycoprotein (MOG), oligodendrocyte-myelin glycoprotein (OMG), cyclic nucleotide phosphodiesterase (CNP), NOGO, myelin protein zero (MPZ), peripheral myelin protein 22 (PMP22), protein 2 (P2), galactocerebroside (GalC), sulfatide and proteolipid protein (PLP).

Subsequent to insult, and after sufficient time for remyelination to occur, fluorescence of the marker proteins may be detected using in vitro or in vivo methods known in the art for detection of fluorescence in small animals. In vivo fluorescence can be detected and/or quantified utilizing devices available in the relevant art. For example, using pulsed laser diodes and a time-correlated single photon counting detection system coupled to a visualization system can detect the level of fluorescence emission from tissues. (Gallant et al., Annual Conference of the Optical Society of America (2004).; Contag et al, Mol. Microbiol. 18:593-603 (1995; Schindehutte et al., Stem Cells 23:10-15 (2005)). To avoid a large signal from back-reflected photons at the tissue-air interface, the detection point is located at 3 mm to the right of the source point. Wavelength selection of both laser and filters is dependent on the fluorescent marker of choice. Where biological tissue absorption is low, fluorescent signals from larger tissue depths (e.g., a few to several centimeters depending on laser power) can be detected for in vivo imaging.

Mice to be imaged may be anesthetized with isoflurane/oxyten and placed on the imaging stage. Ventral and dorsal images can be collected for various time points using imaging systems available in the relevant art (e.g., IVIS imaging system, Xenogen Corp., Alameda, Calif.). Fluorescence from various target tissue can be imaged and quantified. For example, signal intensity can be presented in text or figures as a means +/− standard error about the mean. Fluorescence signals can be analyzed by analysis of variance with post hoc t tests to evaluate the difference between fluorescence signal for a given marker at time zero and each subsequent time point.

Fluorescence visualization, imaging or detection can be made using methods known in the art and described herein, supra. Visualization, imaging or detection can be made through invasive, minimally invasive or non-invasive techniques. Typically, microscopy techniques are utilized to detect or image fluorescence from cells/tissue obtained from the transgenic animals, from living cells, or through in vivo imaging techniques. Supra, “General Methodologies”.

Luminescent, fluorescent or bioluminescent signals are easily detected and quantified with any one of a variety of automated and/or high-throughput instrumentation systems including fluorescence multi-well plate readers, fluorescence activated cell sorters (FACS) and automated cell-based imaging systems that provide spatial resolution of the signal. A variety of instrumentation systems have been developed to automate detection including the automated fluorescence imaging and automated microscopy systems developed by Cellomics, Amersham, TTP, Q3DM, Evotec, Universal Imaging and Zeiss. Fluorescence recovery after photobleaching (FRAP) and time lapse fluorescence microscopy have also been used to study protein mobility in living cells.

Visualizing fluorescence (e.g., marker gene encoding a fluorescent protein) can be conducted with microscopy techniques, either through examining cell/tissue samples obtained from an animal (e.g., through sectioning and imaging using a confocal microscope), examining living cells or detection of fluorescence in vivo. Visualization techniques include, but are not limited to, utilization of confocal microscopy or photo-optical scanning techniques known in the art. Generally, fluorescence labels with emission wavelengths in the near-infrared are more amenable to deep-tissue imaging because both scattering and autofluorescence, which increase background noise, are reduced as wavelengths increase. Examples of in vivo imaging are known in the art, such as disclosed by Mansfield et al., J. Biomed. Opt. 10:41207 (2005); Zhang et al., Drug Met. Disp. 31:1054-1064 (2003); Flusberg et al., Nat. Meth. 2:941-950 (2005); Mehta et al., Curr Opin Neurobiol. 14:617-628 (2004); Jung et al., J. Neurophysiol. 92:3121-3133 (2004); U.S. Pat. Nos. 6,977,733 and 6,839,586, each disclosure of which is herein incorporated by reference.

One example of an in vivo imaging process comprises one week before the in vivo imaging experiment, the dorsal hair in telogen is depilated (about 2.5 cm×2.5 cm area) using a depilatory agent (Nair, Carter-Wallace Inc.). On the day of the imaging experiment, the mouse is anaesthetized and placed with its dorsal skin on a microscope coverslip on the microscope stage. The depilated area of the epidermis is illuminated by a 50W mercury lamp and scanned using an inverted laser scanning confocal fluorescent microscope (Zeiss LSM 510) with a ×10 objective and an LP 520 emission filter (Zeiss). A laser, such as Argon laser (488 nm) and a ×10 objective can image fluorescence emissions, progressively more effectively from deep tissue up to the epidermal cells. By utilizing enhanced emitters or longer wavelength emitters, the sensitivity for deeper tissue imaging can be enhanced. Alternatively small animals, such as mice can easily be scanned/imaged utilizing various different positions (e.g., dorsal, ventral, etc.). In vivo imaging has been effective even with deep tissue regions, such as liver. (e.g., Zhang et al., supra).

Cell/tissue sections mounted with Vectashield mounting medium with DAPI (Vector Laboratories) can be visualized with a Zeiss Axioplan fluorescence microscope. Images can be captured using a Photometrics PXL CCD camera connected to an Apple Macintosh computer using the Open Lab software suite. Fluorescence of different wavelengths is detected and quantified by counting positive cells within the median of the corpus callosum, confined to an area of 0.04 mm². Additional methods for detecting and measuring levels of fluorescence from tissue/cell in vitro utilizing fluorescence or confocal microscopy are known in the art and can be utilized in detecting or measuring fluorescence from one or more marker proteins disclosed herein above.

In another example, neural cells can be imaged with an Axiovert S100 TV inverted microscope fitted with Ludl filter wheels (CarIZeiss, Thomwood, N.Y., USA) in the epifluorescence excitation and emission paths, and a cooled charge-coupled device (CCD) camera (Micro-MAXO; Roper Scientific, Trenton, N.J., USA) can be used to collect the images. Specific excitation and emission filters and a common dichroic element can be used to isolate the signals of the two different fluorescent proteins (HQFITC and Texas Red excitation and emission filters, and FITC/Texas Red V3 dichroic; Chrorna Technology, Brattleboro, Vt., USA). The filter wheels and camera can be controlled by software (e.g., IPLabs software, Scanalytics, Fairfax, Va., USA). Sets of the red and green fluorescent images can be collected to analyze the relative percentage of cells that have red or green fluorescence. The images may be analyzed and prepared for publication with IPLabs and Adobe InDesign software. Manipulations of the images may be confined to merging the grayscale images of the red and green fluorescent proteins to create RGB color files, adjusting the brightness/contrast of the final printouts to match most closely what is observed through the microscope and adding lettering and a scale bar. Fluorescence microscopy apparatus are known in the art and commercially available. See, e.g., website at <confocal-microscopy.com/website/sc_llt.nsf.>

In an alternative embodiment, fluorescence detection is directly from the retina or cornea. The retinal site is a non-invasive locus for study of systemic toxicity. The cornea is particularly well suited to assessing toxicity of substances applied directly to an organ containing glial cells without invading the body. Therefore, fluorescence emitted from neural cells differentially expressing a marker protein can be detected by using confocal microscopy of the retina or cornea by training the laser beam onto the desired region and detecting the level of fluorescence emitted.

Moreover, demyelination/remyelination phenomena can be observed by immunohistochemical means or protein analysis known in the art. For example, sections of the test animal's brain can be stained with antibodies that specifically recognize an oligodendrocyte marker. In another aspect, the expression levels of oligodendrocyte markers can be quantified by immunoblotting, hybridization means, and amplification procedures, and any other methods that are well-established in the art. e.g., Mukouyama et al., Proc. Natl. Acad. Sci. 103:1551-1556 (2006); Zhang et al., supra; Girard et al., J. Neurosci. 25:7924-7933 (2005); and U.S. Pat. Nos. 6,909,031; 6,891,081; 6,903,244; 6,905,823; 6,781,029; and 6,753,456, the disclosure of each of which is herein incorporated by reference.

In another aspect, cell/tissue from the central or peripheral nervous system can be excised and processed for the protein, e.g., tissue is homogenized and protein is separated on an SDS-10% polyacylamide gel and then transferred to nitrocellulose membrane to detect marker proteins. Fluorescent protein levels can be detected utilizing primary antibody/antisera (e.g., goat polyclonal raised against a particular marker protein; BD Gentest, Woburn, Mass.) and peroxidase-conjugated secondary antibody (e.g. rabbit anti-goat IgG, Sigma-Aldrich). Chemiluminescence is detected using standard reagents available in the art to detect and determine levels of fluorescence marker proteins in tissue samples.

Therefore, if a candidate therapeutic/drug is being assayed in one or more methods of the invention, then it can be determined if there is an overall difference in response to the drug compared at different time points, as well as compared to reference or controls. In summary, by detecting and quantifying expression of a marker which is differentially expressed in a single subpopulation of glial cells (e.g., progenitor oligodendrocytes), such as using the transgenic animal in the foregoing example it to obtain various data, it may be determined whether remyelination is occurring post insult and whether a candidate agent modulates such remyelination and to what degree. Of course, one of ordinary skill in the art will recognize that the foregoing is merely one example for utilizing the remyelination model system of the present invention.

Cell-Based Assays:

In some aspects of the present invention the transgenic animals of the invention can be the source for cell/tissue culture. For example, the practice of the invention may involve cell-based assays for providing a comparison of the expression of a gene or gene product or the activity of said gene product in a test neural cell (e.g., transgenic oligodendrocyte or Schwann cell) relative to a control cell. The test neural cell used for this invention can be isolated from central nervous system (CNS) or peripheral nervous system (PNS), and includes cell culture derived from the cells of the transgenic animals, the progeny thereof, and section or smear prepared from the source, or any other samples of the CNS or PNS, for example, oligodendrocytes, Schwann cells, or neurons; the mature or immature cells. Where desired, one may choose to use enriched cell cultures that are substantially free of other neural cell types such as neurons, microglial cells, and astrocytes. Various methods of isolating, generating or maintaining matured oligodendrocytes and Schwann cells are known in the art (Baerwald et al., J. Neurosci. Res. 52:230-239 (1998); Levi et al., J Neurosci. Meth. 68:21-26 (1998)) and are exemplified herein.

In one embodiment, the present invention provides a method of identifying a candidate biologically active agent that modulates remyelination. The method involves the steps of (a) obtaining or isolating neural cells from transgenic animals of the present invention capable of neural cell-differential expression of marker proteins and culturing such cells; (b) contacting a candidate agent with the cultured neural cell; (c) detecting an altered expression of a gene or gene product or an altered activity of said gene product relative to a control cell, said gene or gene product being correlated to modulation of remyelination; and (d) selecting said agent as a candidate if the level of expression of said gene or gene product modulated relative to said control cell.

In another embodiment, the present invention provides a method of identifying a biologically active agent that promotes neuronal remyelination. The method comprises the steps of (a) obtaining, isolating and culturing neural cells from a demyelinated lesion present in a transgenic animal of the present invention; (b) contacting a candidate biologically active agent with the cultured neural cells; and (b) detecting an altered expression of a gene or gene product or an altered activity of said gene product relative to a control cell, said gene or gene product being correlated to remyelination; and (c) selecting said agent as a candidate if the level of expression of said gene or gene product, or the level of activity of said gene product, is increased relative to said control cell. In certain embodiments, it may be preferable to employ myelinating cells from young subjects whose nervous systems are actively undergoing myelination. In other embodiments, it may be preferable to use remyelinating cells derived from adult oligodendrocyte precursors in demyelinated lesions, including but not limited to lesions inflicted by pathogens or physical injuries, and lesions caused by toxic agents such as cuprizone.

Alternatively, neural cells can be isolated and cultured from transgenic animals of the invention that contain a single “knock-in” gene (e.g., PDGFα/recombinase or PLP/stoplight). Such neural cells can be genetically modified. For example, new variants of neural cells can be generated by introducing into the cell a genetic vehicle comprising a desired gene construct. For example, neural cells obtained from a transgenic animal comprising the inducible PDGFα/Recombinase gene construct, can be transfected with a genetic vehicle comprising the PLP/Stoplight construct. Alternatively, neural cells obtained from a transgenic animal can be transfected with a bicistronic genetic vehicle comprising an inducible gene encoding a desired product as well as an expression construct encoding one or more reporter genes. In addition, isolated cells can be co-transfected with multiple genetic vehicles (e.g., two vectors each of which comprises gene constructs encoding a desired product and gene constructs encoding one or more reporter genes).

The selection of an appropriate control cell or tissue is dependent on the test cell or tissue initially selected and its phenotypic or genotypic characteristic which is under investigation. Whereas the test remyelinating cell is contacted with a test compound, then a control cell or tissue may be a non-treated counterpart. Whereas the test remyelinating cell is a test cell detected post demyelination, the control cell may be a non-treated counterpart. It is generally preferable to analyze the test cell and the control in parallel.

For the purposes of this invention, a biologically active agent effective to modulate neuronal remyelination is intended to include, but not be limited to, a biological or chemical compound such as a simple or complex organic or inorganic molecule, peptide, peptide mimetic, protein (e.g. antibody), liposome, small interfering RNA, or a polynucleotide (e.g. anti-sense).

A vast array of compounds can be synthesized, for example polymers, such as polypeptides and polynucleotides, and synthetic organic compounds based on various core structures, and these are also contemplated herein. In addition, various natural sources can provide compounds for screening, such as plant or animal extracts, and the like. It should be understood, although not always explicitly stated, that the active agent can be used alone or in combination with another modulator, having the same or different biological activity as the agents identified by the subject screening method.

When the biologically active agent is a composition other than naked RNA, the agent may be directly added to the cell culture or added to culture medium for addition. As is apparent to those skilled in the art, an “effective” amount must be added which can be empirically determined. When the agent is a polynucleotide, it may be introduced directly into a cell by transfection or electroporation. Alternatively, it may be inserted into the cell using a gene delivery vehicle or other methods as described above.

A wide variety of labels suitable for detecting protein levels are known in the art. Non-limiting examples include radioisotopes, enzymes, colloidal metals, fluorescent compounds, bioluminescent compounds, and chemiluminescent compounds.

Candidate biologically active agents identified by the subject methods can be broadly categorized into the following two classes. The first class encompasses agents that when administered into a cell or a subject, reduce the level of expression or activity of a marker gene specific for remyelinating neural cells. The second class includes agents that augment the level of expression or activity of a marker gene specific for remyelinating neural cells.

As discussed in the sections above, these cells are particularly useful for conducting cell-based assays for elucidating the molecular basis of neuronal remyelination conditions, and for assaying agents effective for inhibiting neuronal demyelination or promoting remyelination.

Therefore, if a candidate therapeutic/drug is being assayed in one or more methods of the invention, then it can be determined if there is an overall difference in response to the drug compared at different time points, as well as compared to reference or controls. In summary, by detecting and quantifying expression of a marker which is differentially expressed in a single subpopulation of glial cells (e.g., progenitor oligodendrocytes) the transgenic animal in the foregoing example is used to obtain various data, which include whether remyeliantion is occurring post insult and whether a candidate agent modulates such remyelination and to what degree. Of course, one of ordinary skill in the art will recognize that the foregoing is merely one example for utilizing the remyelination model system of the present invention.

Cell Fate Mapping:

In another aspect, the gene constructs of the invention can be utilized to track or define cell/tissue lineage. Isolation or identification of defined cell populations from a certain cell lineage or tissue would be a pre-requisite for analysis of cell differentiation and development. For example, tracing lineage is important in designing applications requiring analysis of stem cell-derived cells, such applications including tracing cell differentiation from a selected embryonic cell clone, or assessing expression in vivo where mice comprising the stoplight construct with an appropriate promoter, is crossed with Cre-expressing mice. In this latter scenario, Cre is conditionally expressed in a specific cell during development and derivatives from this cell are detected (via e.g. EGFP). In other words, the stoplight mouse with a tissue specific promoter can be crossed with conditional-Cre mice, for example, to track neuroectodermal derivatives using Wnt1-Cre, mesodermal derivatives using RARβ2-Cre, or in endocrine pancreas of endodermal origin using Pax4-Cre, as well as the PDGFα-receptor/Cre described above.

Therefore, in some embodiments, the promoter element for the constructs—PDGFα-receptor/CreER^{T2} and/or PLP/loxP-mCherry-loxP-EGFP—may be replaced by constitutive promoters that will express the “floxed” reporter in, for example, embryonic stem cells and progeny cells and are subsequently identified via Cre recombination, e.g., inducing Cre expression that results in the second fluorescent marker in the stoplight construct to be expressed. In additional embodiments, the stoplight and Cre constructs can be modified to contain any cell- or tissue-specific promoters as desired. Such promoters are known in the art. As such the Cre-stoplight system can be utilized to trace lineage of cells via the conditional expression of Cre and the resulting differential expression of one of two fluorescent markers. Thus, the conditional expression system allows labeling of single cells (e.g., mcherry expression) and subsequently tracing their clonal lineage, whereby the Cre recombination allows genetically tagging derivative cells from a stem cell clone (e.g., EGFP expression).

Promoter elements that can be utilized in the Cre or stoplight constructs include but are not limited to promoters and/or enhancers which are specifically active in dopaminergic, serotoninergic, GABAergic, cholinergic or peptidergic neurons and sub-populations thereof; neural cells, particularly glial cells, more particularly, oligodendrocytes, astrocytes and sub-populations thereof; neurotransmitter-specific receptors, ion channels, receptors involved in ion channel gating, cytokines, growth factors and hormones, and those known in the art or disclosed in the disclosures of Patterson et al., J. Biol. Chem. 270:23111-23118 (1995); U.S. Pat. Nos. 6,472,520, 7,022,319, 7,033,595, U.S. Pat. Application 20060052327, 20060040386, 20060034767, 20060030541, all of which are incorporated herein by reference.

In other embodiments, cell fate mapping can be effected utilizing different progenitor cells to define regeneration occurring in a tissue/organ, including muscle, kidney, liver, spleen, heart, lungs, brain, central nervous system, peripheral nervous system, optic nerve, eye, retina, lymphatic tissue, thymus, thyroid, parathyroid, gastrointestinal tract, stomach, prostate, testis, ovaries, dermis, skin, reproductive organ, endothelial cells or vasculature. For example, floxed constructs comprising cell/tissue specific promoters described herein and known in the art, can be utilized to define cell lineage observed in cell/tissue regeneration occurring in cell culture or in vivo. In other words, various promoter/enhancers elements can be incorporated into the floxed vector and/or a vector expressing a recombinase specific for flanking sequences present on the floxed vector. Examples of site-specific recombinases are known in the art and described herein.

Therefore, in some embodiments, wherein regeneration of cells/tissue occurring in a tissue/organ, including muscle, kidney, liver, spleen, heart, lungs, brain, central nervous system, peripheral nervous system, optic nerve, eye, retina, lymphatic tissue, thymus, thyroid, parathyroid, gastrointestinal tract, stomach, prostate, testis, ovaries, dermis, skin, reproductive organ, endothelial cells or vasculature is detected by calorimetric microscopy, cell lineage can be determined based on a different detectable signals observed (e.g., detectable signal encoded on floxed construct).

Non-limiting examples of promoters that can be utilized in the methods of the invention include promotes from genes for uncoupling protein 3, α human folate receptor, whey acidic protein, prostate specific promoter and as also disclosed in U.S. Pat. No. 6,313,373 and as disclosed online at <biobase/de/pages/products/transpor.html>, which is a database with over 15,000 different promoter sequences classified by genes/activity; and Chen et al. Nuc. Acids. Res. 2006, 34: Database issue, D104-107; See also, the website <tiprod.cbi.pku.edu.cn:8080/index>, which also lists promoters of genes specific to certain cell/tissue.

In another aspect of the present invention, promoters used may be specific to neurons after axotomy, thus, one may monitor axonal repair after injury. For example, the Cre recombinase may be under the control of a promoter of a gene induced after axotomy. Examples of such genes include retinoic acid binding proptein 2, retinol-binding protein 1, tumor-associated glycoprotein pE4, endothelial monocyte-activating polypeptide, neurolysin (metallopeptidase M3 family), GADD 45, Moesin, SPRR1, sphingosine kinase 1, and galanin (Wang et al., Disease Gene Candidates Revealed by Expression Profiling of Retinal Ganglion Cell Development, J. Neurosci. 27 in press (2007)). The Cre recombinase may be active when expression of Cre is induced by the promoter of the aforementioned genes. The stoplight construct may be under the control of a neuron specific promoter, such as that of Tau. In this example, the first marker protein is expressed in neuron cells, and after injury, Cre recombinase expression is induced, the first maker gene is excised (e.g. mcherry), and the second marker protein (e.g. EGFP) is expressed (e.g. FIG. 13).

Alternatively, an inducible Cre such as CreER^{T2} is under the control of a neuronal precursor cell promoter, such as Nestin and the stoplight cassette is under the control of a promoter of a gene with increased expression during development and after axon injury, such as Best 5, TIMP 1, methallothionein, ATF 3, monoglyceride lipase, PTP non-receptor Type 5, LPS-induced TNF-factor, FXVD ion transport reg 7, and proline-transporter (Wang et al., Disease Gene Candidates Revealed by Expression Profiling of Retinal Ganglion Cell Development, J. Neurosci. 27 in press (2007)). Thus, expression of the first marker protein is in developing axons. Cre is induced with an exogenous agent, such as tamoxifen for CreER^{T2}, and the cells subjected to injury. As a result, the first marker gene is excised, and regrowing axons express the second marker protein (FIG. 14).

Of course, alternatively or in addition to cell/tissue-specific promoters, it should be understood that regulatable promoters known in the art can also be utilized. For example, the construct encoding a recombinase protein can be operably linked to a regulatable promoter, such as a tet-responsive promoter. Therefore, where and when desired an inducible agent (e.g., tetracycline or analog thereof) can be administered to cells or a subject to induce expression of the recombinase protein which will then act upon the floxed construct, which can also comprise a tissue/specific or regulatable promoter, thus resulting in differentiae expression of a detectable signal (e.g., EGFP or Chemy Red). Examples of regulatable promoters include but are not limited to MMTV, heat shock 70 promoter, GAL1-GAL10 promoter, metallothien inducible promoters (e.g., copper inducible ACE1), hormone response elements (e.g., glucocorticoid, estrogen, progestrogen), and those known in the art to function as regulatable promoter in mammalian cells, in culture or in vivo.

Ex Vivo Applications:

In some aspects of the invention, transgenic cells can be obtained from the transgenic animals of the invention, cultured and expanded, transduced with a gene encoding a target protein, and implanted or reintroduced into the source animal or some other animal. In such ex vivo methods, the transgenic cells can be transfected with a gene encoding a biologically active agent (e.g., gene encoding a test product) that can be inducibly produced for example, so as to assay the test gene/protein for modulation of marker gene expression/production. Such modulation can be assayed in cell culture as described herein above. Alternatively, transduced cells are reintroduced into the subject animal, where marker gene expression can be assayed and compared to a control or reference, where the cells transplanted are not transduced, do not express a vector-borne product of interest, express a vector-borne product of interest in a time controlled manner (e.g., inducible expression) or express the product of interest constitutively (e.g., CMV promoter).

For example, glial cells (e.g., oligodendrocytes or Schwann cells) can be derived from nerve biopsies. Cells can be expanded in culture (e.g., utilizing proliferating medium composed of DMEM containing 10% heat-inactivated fetal bovine serum (FBS) and supplemented with antibiotics, recombinant Neu differentiation factor (NDF), insulin and forskolin (1 ug/ml)). Furthermore, cells can be sorted from non-transgenic nerve cells utilizing the fluorescence labels provided by the transgene(s) (e.g., FACS). For transduction, cells can be tranfected with various vector vehicles known in the art that will deliver a product of interest.

Pharmaceutical Compositions of the Present Invention:

The one or more methods of the invention disclosed herein can be utilized to select a biologically active agent that can subsequently be implemented in treatment of demyelination. The selected biologically active agents effective to modulate remyelination may be used for the preparation of medicaments for treating neuronal demyelination disorders. In certain embodiments, the demyelination disorder referred herein is multiple sclerosis. In other embodiments, the demyelination disorder is selected from the group consisting of Progressive Multifocal Leukoencephalopathy (PML), Encephalomyelitis, Central Pontine Myelolysis (CPM), Anti-MAG Disease, Leukodystrophies: Adrenoleukodystrophy (ALD), Alexander's Disease, Canavan Disease, Krabbe Disease, Metachromatic Leukodystrophy (MLD), Pelizaeus-Merzbacher Disease, Refsum Disease, Cockayne Syndrome, Van der Knapp Syndrome, and Zellweger Syndrome, Guillain-Barre Syndrome (GBS), chronic inflammatory demyelinating polyneuropathy (CIDP), and multifocual motor neuropathy (MMN).

In one aspect, an identified/selected biologically active agent of this invention can be administered to treat neuronal demyelination inflicted by pathogens such as bacteria and viruses. In another aspect, the selected agent can be used to treat neuronal demyelination caused by toxic substances or accumulation of toxic metabolites in the body as in, e.g., central pontine myelinolysis and vitamin deficiencies. In yet another aspect, the agent can be used to treat demyelination caused by physical injury, such as spinal cord injury. In still yet another aspect, the agent can be administered to treat demyelination manifested in disorders having genetic attributes, genetic disorders including but not limited to leukodystrophies, adrenoleukodystrophy, degenerative multi-system atrophy, Binswanger encephalopathy, tumors in the central nervous system, and multiple sclerosis. The agent may also be administered to treat diseases that affect remylination, or hypoxic conditions or injury that affect remyelination, such as ischemia, stroke, or Alzheimers.

The identified/selected biologically active agent of the invention may also be delivered with, prior to, or subsequent to, other products of interest that may be selected from, but not limited to: a growth factor, cytokine, nerve growth factor, anti-sense RNA, siRNA, immuno-suppressants, anti-inflammatories, anti-proliferatives, anti-migratory agents, anti-fibrotic agents, pro-apoptotics, antibodies, anti-thrombotic agents, anti-platelet agents, IIbIIIIa agents, angiogenic factors, anti-angiogenic factors, antiviral agents, nerve growth factor, NGF family of proteins, NGF, Beta-NGF, Neurotrophin-3 precursor (NT-3), HDNF, Nerve growth factor 2 (NGF-2), Brain-derived neurotrophic factor (BDNF), Neurotrophin-5 (NT-5), Neurotrophin-4 (NT-4), or precursors and combinations thereof.

Various delivery systems are known and can be used to administer a biologically active agent of the invention, e.g., encapsulation in liposomes, microparticles, microcapsules, expression by recombinant cells, receptor-mediated endocytosis (see, e.g., Wu and Wu, J. Biol. Chem. 262:4429-4432 (1987)), construction of a therapeutic nucleic acid as part of a retroviral or other vector, etc. Methods of delivery include but are not limited to intra-arterial, intra-muscular, intravenous, intranasal, and oral routes. In a specific embodiment, it may be desirable to administer the pharmaceutical compositions of the invention locally to the area in need of treatment; this may be achieved by, for example, and not by way of limitation, local infusion during surgery, by injection, or by means of a catheter. In certain embodiments, the agents are delivered to a subject's nerve systems, preferably the central nervous system. In another embodiment, the agents are administered to neural tissues undergoing remyelination.

Administration of the selected agent can be effected in one dose, continuously, or intermittently throughout the course of treatment. Methods of determining the most effective means and dosage of administration are well known to those of skill in the art and will vary with the composition used for therapy, the purpose of the therapy, the target cell being treated, and the subject being treated. Single or multiple administrations can be carried out with the dose level and pattern being selected by the treating physician.

The preparation of pharmaceutical compositions of this invention is conducted in accordance with generally accepted procedures for the preparation of pharmaceutical preparations. See, for example, Remington's Pharmaceutical Sciences 18th Edition (1990), E. W. Martin ed., Mack Publishing Co., PA. Depending on the intended use and mode of administration, it may be desirable to process the active ingredient further in the preparation of pharmaceutical compositions. Appropriate processing may include mixing with appropriate non-toxic and non-interfering components, sterilizing, dividing into dose units, and enclosing in a delivery device.

Pharmaceutical compositions for oral, intranasal, or topical administration can be supplied in solid, semi-solid or liquid forms, including tablets, capsules, powders, liquids, and suspensions. Compositions for injection can be supplied as liquid solutions or suspensions, as emulsions, or as solid forms suitable for dissolution or suspension in liquid prior to injection. For administration via the respiratory tract, a preferred composition is one that provides a solid, powder, or aerosol when used with an appropriate aerosolizer device.

Liquid pharmaceutically acceptable compositions can, for example, be prepared by dissolving or dispersing a polypeptide embodied herein in a liquid excipient, such as water, saline, aqueous dextrose, glycerol, or ethanol. The composition can also contain other medicinal agents, pharmaceutical agents, adjuvants, carriers, and auxiliary substances such as wetting or emulsifying agents, and pH buffering agents.

### EXAMPLES

### Example 1

### Generation of a Targeting Construct for CreER^{T2} Knockin into the Mouse Gene that Encodes the Platelet-Derived Growth Factor Receptor-Alpha (PDGFR-α)

A ˜10.2 kb region used to construct the targeting vector was first sub cloned from a positively identified C57BL/6 BAC clone using a homologous recombination-based technique. The region was designed such that the short homology arm (SA) extends 1.6 kb to 5′ of FLP recombinase target (FRT) sequences that flanked the Neo and Cre-ERT2 cassette. The long homology arm (LA) ends on the 3′ side of FRT flanked Neo and Cre-ERT2 cassette and is ˜8.6 kb long. The exon 1 of this gene was replaced with the FRT flanked Neo and Cre-ERT2 cassette (FIG. 4).

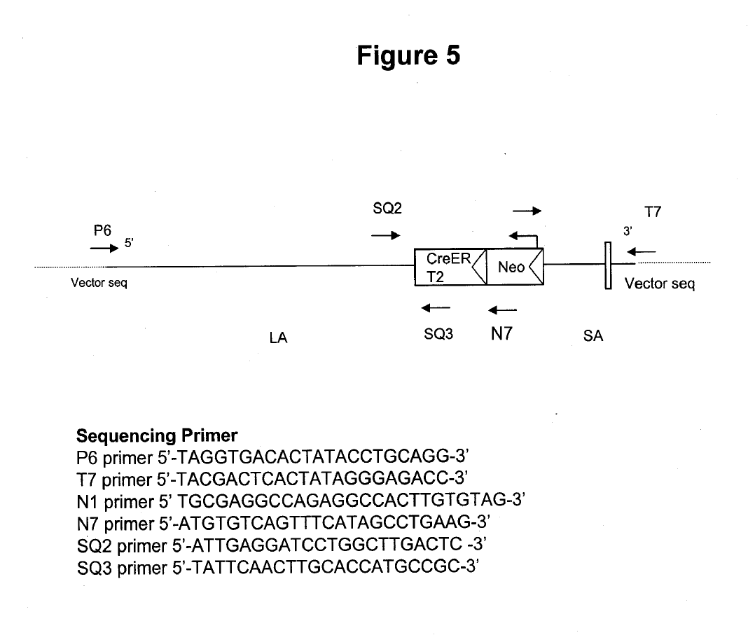

The targeting vector was confirmed by restriction analysis after each modification step and by sequencing using primers designed to read from the selection cassette into the 3′ end of the LA (N2) and the 5′ end of the SA (N1), or from primers that anneal to the vector sequence, P6 and T7, and read into the 5′ and 3′ ends of the BAC sub clone (FIG. 5).

The BAC was sub cloned into a ˜2.4 kb pSP72 (Promega) backbone vector containing an ampicillin selection cassette for retransformation of the construct prior to electroporation. A FRT-flanked Neomycin cassette was inserted into the gene as described in the project schematic (FIG. 4). The targeting construct can be linearized using NotI prior to electroporation into ES cells. The total size of the targeting construct (including vector backbone) is ˜17.6 kb.

### Example 2

### Electroporation and Screening for PDGFR-α/CreER^{T2} Recombinant Clones

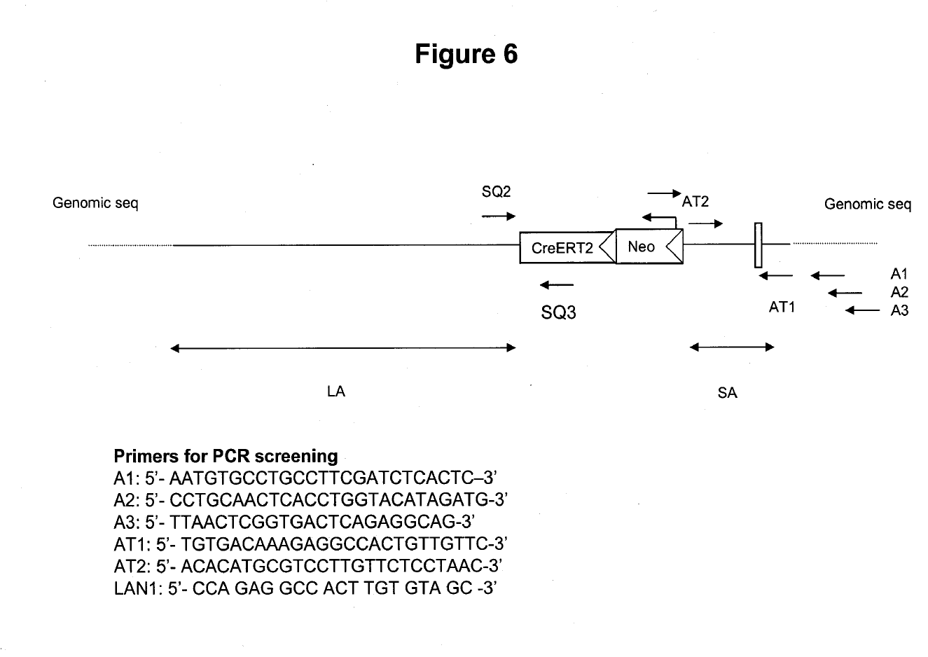

Ten micrograms of the targeting vector in Example 1 was linearized by NotI and then transfected by electroporation of iTL IC1 C57BL/6 embryonic stem cells. After selection in G418 antibiotic, surviving clones were expanded for PCR analysis to identify recombinant ES clones. Primers, A1, A2 and A3 were designed downstream (3′) to the short homology arm (SA) outside the region used to generate the targeting construct (FIG. 6). PCR reactions using A1, A2 or A3 with the LAN1 primer at the 5′ end of the Neo cassette amplify 1.9, 1.9, and 2.0 kb fragments, respectively. The control PCR reaction was done using primer pair AT1 and AT2, which is at the 5′ end of the SA inside the region used to create the targeting construct. This amplifies a band of 1.5 kb. The PCR parameters were 95° C. for 30 seconds, 64° C. for 30 seconds, 72° C. for 150 seconds for 35 cycles.

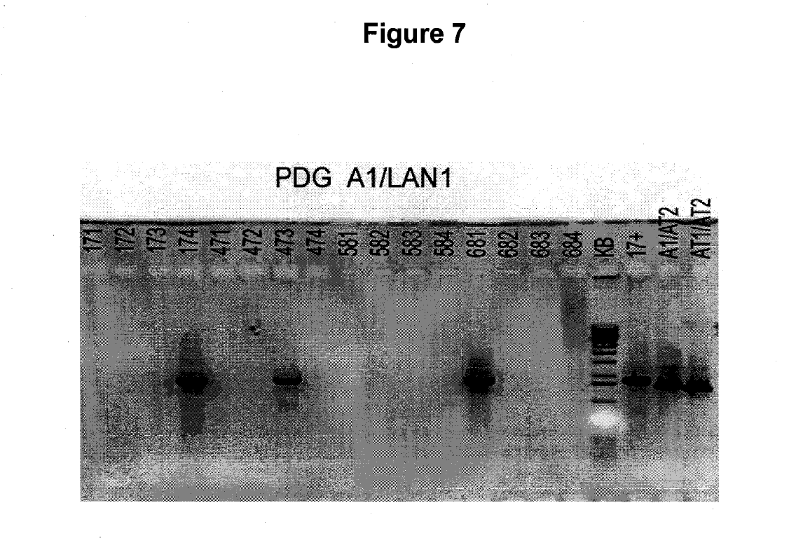

Individual clones were screened with A1/LAN1 primers. Recombinant clones were identified by a 1.9 Kb PCR fragment. The positive controls were positive pooled samples and indicated as (+) (FIG. 7). PCR reaction controls were done with screening and internal primers.

The recombinant clones were expanded and confirmed. ES cells from the positive clones have been injected into the blastocoel cavity of BALB/c preimplantation embryos. Several chimeric animals have been generated. These mice are mated to identify germ-line transmitants.

### Example 3

### Generation of a Targeting Construct for Reporter Genes

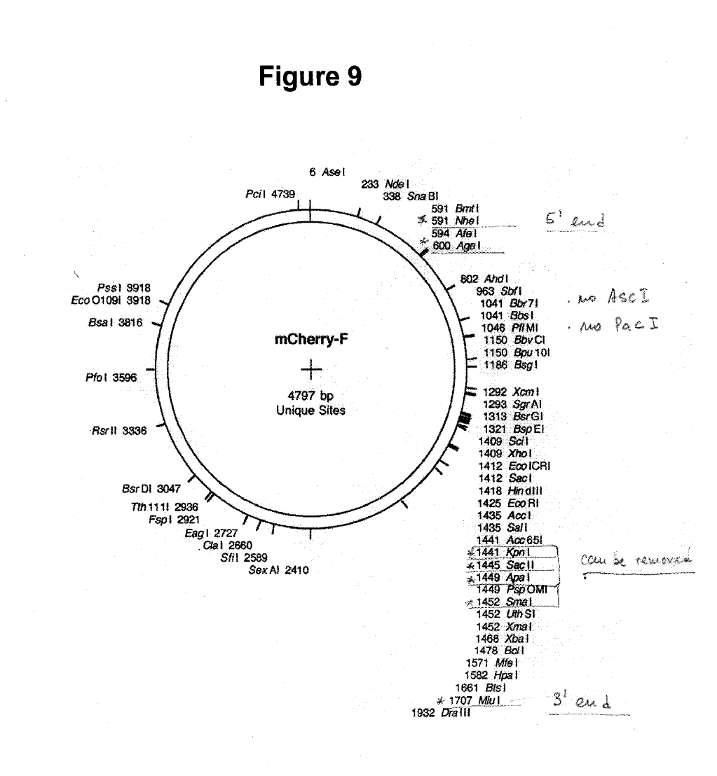

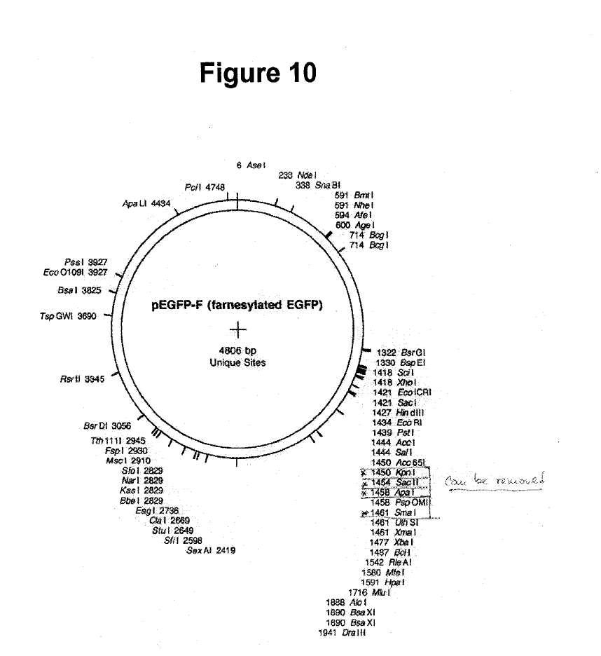

The targeting construct for the reporter gene is depicted in FIG. 8. Restriction enzyme sites from mCherry-F (FIG. 9) and pEGFP-F (FIG. 10) plasmids were removed by digesting the plasmids with Acc65I and SmaI. The DNA was then gel extracted. Blunt end ligations (using Klenow) were performed to fill in the Acc65I digestion sites, and the DNA was gel extracted. The blunt-ended vectors were ligated, and transformed into E. coli DH5α. Plasmid isolation was performed on individual colonies. Plasmid preps that had the correct digest patterns were sequenced with primers REREM-R, Chemy-F and EGFP-F to verify deletion of the restriction enzyme sites. The vectors were named mCherry-F-REREM and pEGFP-F-REREM.

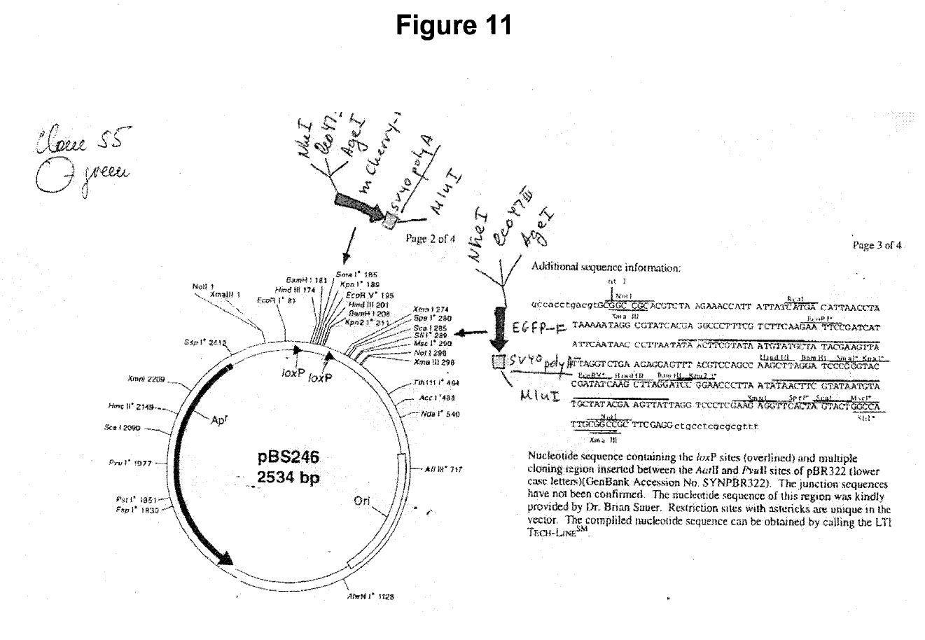

The mCherry-F-REREM and EGFP-F-REREM were then cloned into pBS246 (FIG. 11). Both mCherry-F-REREM and pEGFP-F-REREM were digested with AgeI and MluI, and the ˜1.1 kb DNA fragments were gel extracted. pBS246 was digested with SmaI and treated with phosphatase (CIP). The DNA was gel extracted. Digested mCherry-F-REREM and digested pBS246 were ligated, and transformed into E. coli DH5α. Plasmid isolation was performed on individual colonies. Plasmid preps that had the correct digest patterns were sequenced with primers Chemy-F and Chemy-Lox-R to verify that mCherry-F-REREM had been correctly inserted between the LoxP sites in pBS246. The 3.6 Kb vector was named pBS246-mCherry. The pBS246-mCherry was digested with SpeI, treated with phosphatase, and gel extracted. Digested EGFP-F-REREM was ligated to digested pBS246-mCherry, and transformed into E. coli DH5α. Plasmid isolation was performed on individual colonies. Plasmid preps that had the correct digest patterns were sequenced with primers Chemy-F, Chemy-Lox-R, EGFP-F and EGFP-R to verify that both mCherry and EGFP were correctly inserted in pBS246. The 4.8 Kb vector was named pBS246-mCherry-EGFP.

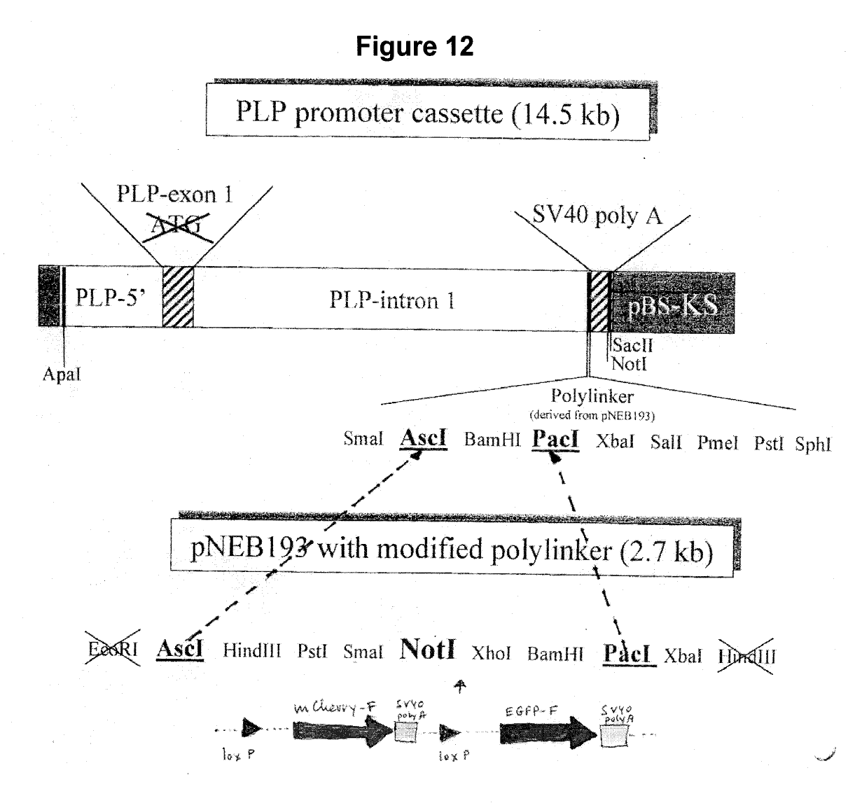

To generate restriction enzyme sites AscI and PacI for insertion into the PCP promoter cassette (FIG. 12), pBS246-mCherry-EGFP was digested with SspI and NotI, and the 3.7 kb DNA insert was gel extracted. pNEB193 was digested with SmaI and NotI, and gel extracted. The Lox-mCherry-Lox-EGFP insert was ligated to digested pNEB193, and transformed into E. coli DH5α. Plasmid isolation was performed on individual colonies. Plasmid preps that had the correct digest patterns were sequenced with primers Chemy-F, Chemy-R, EGFP-F and EGFP-R to verify that Lox-mCherry-Lox-EGFP had been correctly inserted into pNEB193. The 6 Kb vector was named pNEB-mCherry-EGFP. pNEB-mCherry-EGFP was digested with AscI and PacI, and gel extracted.

To construct PLP-neo-SC101ori for the targeting vector, the neo cassette, Lox-FRT-neo/Kan-Lox-FRT, was digested with BsiWI, blunt ended, and gel extracted. pBR322 was digested with EcoRV and treated with phosphatase. The digested neo-cassette was ligated to pBR322 and transformed into E. coli DH5α. Plasmid isolation was performed on individual colonies, and the neo-cassette was confirmed by digestion. The 6.2 Kb vector was named pBRG. To remove the 3′ LoxP site from the neo-cassette, pBRG was digested with SacII and EcoRV, blunt-ended, and gel extracted, ligated, and transformed. Plasmid isolation was performed on individual colonies. Plasmid preps that had the correct digest patterns were sequenced with primers Neo-REREM-F, and N1 to confirm removal of the 3′ LoxP site from the neo-cassette. The vector was named pGLOXOUT. Primers were designed to amplify the SC101 ori. The primers were engineered to add an NheI site to the 5′ end of the sequence, and an NruI site to the 3′ end. The SC101 ori was PCR amplified, and gel extracted. The PCR product was digested with NheI and NruI, and gel extracted. pGLOXOUT was digested with NheI and NruI, and gel extracted. The PCR product was ligated to pGLOXOUT, and transformed. Plasmid isolation was performed on individual colonies, and the addition of the SC101 ori was confirmed by digestion. The vector was named pGLOXOUT-SCORI. Primers were designed to amplify the neo-cassette +SC101 ori from pGLOXOUT-SCORI. The forward primer contained 20 bp homology to the neo-cassette (2 bp downstream of the 5′ LoxP site, this removed the last LoxP site from the neo-cassette) and 60 bp homology to the PLP-cassette, downstream of the SacII restriction enzyme site. The reverse primer contained 20 bp of homology to the 3′ end of the SC101 ori and 60 bp of homology to the PLP-cassette, upstream of the ApaI restriction enzyme site. When used in a subsequent recombination step, neo^{R}-SC101 ori will replace AmP^{R} and pUC ori in the PLP-cassette. The PCR product was named G3O2. Plasmid isolation was performed on individual colonies to find PLP-G3O2. Plasmid preps that had the correct digest patterns were sequenced with primers PLP-seq-R, SCORI-R, N1 and Neo-REREM-F to verify that the neo-cassette had been inserted with both flanking FRT sites intact, and that both LoxP sites were absent, as well as to confirm the presence of the SC101 ori, and that the PLP-cassette was still in the correct position. The 14.9 Kb vector was named PLP-G3O2. PLP-G3O2 was digested with AscI and PacI, and gel extracted.

PLP-G3O2 was ligated to the AscI/PacI digested lox-mCherry-EGFP-lox insert, and transformed. Plasmid isolation was performed on individual colonies. Plasmid preps that had the correct digest patterns were digested with ApaI and MluI to remove the transgene. The 18 Kb vector was named PLP-mCherry-EGFP-neo-SCORI. The 15 Kb transgene was gel extracted. Digests were performed to confirm the presence of PLP, mCherry, EGFP, and neo R. The gel extracted DNA was sequenced with primers Neo-FRT-R, neo-REREM-F, Chemy-F, Chemy-Lox-R, EGFP-F, EGFP-R, PLP-seq-F, and PLP-seq-R to confirm the presence and position of FRT-neo-FRT, loxP-mCherry-loxP, EGFP, and PLP.

The sequence confirmed DNA was electroporated into iTL IC1 ES cells. After selection in G418 antibiotic, surviving clones were expanded for PCR analysis to identify recombinant ES clones.

The construct should integrate randomly and positive clones are screened for the integrity of the targeting construct and for copy number. Clones with only one integration event have been identified and have been injected into the blastocoel cavity of BALB/c preimplantation embryos.

### Example 4

### Induction of Demyelination and Evaluation of Remyelination in PLP/loxP-mCherry-loxP-EGFP Mice

The PDGFα receptor/CreER^{T2} mice generated from Example 2 is mated with PLP/loxP-mCherry-loxP-EGFP mice generated in Example 3 to produce double transgenic mice (methods known in the art for producing a double transgenic animal, see e.g. Lin et al., J. Neurosci. 24:10074-10083 (2004)).

The double transgenic mice, PDGFα receptor/CreER^{T2} PLP/loxP-mCherry-loxP-EGFP, should express mCherry in myelinating cells. Prior to demyelination insult, the animals are treated with tamoxifen, to induce Cre recombinase, which should excise the mCherry transgene, in adult oligodendrocyte progenitor cells (FIGS. 1-3). As such, only oligodendrocyte progenitor cells containing the Cre construct will enable expression of EGFP when the oligodendrocyte progenitor cells mature to myelinating cells.

To induce demyelination, mice are fed a diet of milled mouse chow containing 0.2% cuprizone (Sigma-Aldrich, St. Louis, Mich.) for up to 6 weeks. Subsequently, mice are returned to a normal diet for up to 3 weeks to allow remyelination to occur.

Subsequent to insult, and after sufficient time for remyelination to occur, fluorescence is detected using in vitro or in vivo methods known in the art for detection of fluorescence in small animals. Mice anesthetized with isoflurane/oxyten are placed on the imaging stage. Ventral and dorsal images are collected for various time points using imaging systems available in the relevant art (e.g., IVIS imaging system, Xenogen Corp., Alameda, Calif.).

Anesthetized mice are also perfused, through the left cardiac ventricle with 4% paraformaldehyde in 0.1M PBS. The brains are removed, postfixed with paraformaldehyde, cryopreserved in 30% sucrose, embedded in OCT and frozen on dry ice. Frozen sections are cut in a cryostat at a thickness of 10 μm. Coronal sections at the formix region of the corpus callosum corresponding to Sidman sections 241-251 are selected for use, and all comparative analyses are restricted to midline corpus callosum (Sidman et al., Atlas of the Mouse Brain and Spinal Cord, Harvard Univ. Press, Cambridge, Mass. (1971)).

In vivo fluorescence is detected and/or quantified utilizing devices available in the relevant art. Pulsed laser diodes and a time-correlated single photon counting detection system coupled to a visualization system are used to detect the level of fluorescence emission from tissues. (Gallant et al., Annual Conference of the Optical Society of America (2004); Contag et al., Mol. Microbiol. 18:593-603 (1995), Schindehutte et al., Stem Cells. 23:10-15 (2005)). To avoid a large signal from back-reflected photons at the tissue-air interface, the detection point is located at 3 mm to the right of the source point. Wavelength selection of both laser and filters is dependent on the fluorescent marker of choice. Where biological tissue absorption is low, fluorescent signals from larger tissue depths (e.g., a few to several centimeters depending on laser power) are detected for in vivo imaging.

Fluorescence from various target tissue are imaged and quantified. Signal intensity is presented in text or figures as a means +/− standard error about the mean. Fluorescence signals are analyzed by analysis of variance with post hoc t tests to evaluate the difference between fluorescence signal for a given marker at time zero and each subsequent time point.

For immunohistochemistry, frozen sections are treated with −20° C. acetone, blocked with PBS containing 10% NGS and 0.1% Triton X-100 and incubated overnight with the primary antibody diluted in blocking solution. Appropriate fluorochrome- or enzyme-labeled secondary antibodies (Vector Laboratories, Burlingame, Calif.) are used for detection. Cell/tissue sections are mounted with Vectashield mounting medium with DAPI (Vector Laboratories) and visualized with a Zeiss Axioplan fluorescence microscope. Images are captured using a Photometrics PXL CCD camera connected to an Apple Macintosh computer using the Open Lab software suite. Fluorescence of different wavelengths is detected and quantified by counting positive cells within the median of the corpus callosum, confined to an area of 0.04 mm². Additional methods for detecting and measuring levels of fluorescence from tissue/cell in vitro utilizing fluorescence or confocal microscopy are known in the art and can be utilized in detecting or measuring fluorescence from one or more marker proteins disclosed herein above.

In addition to fluorescence detection via microscopy, cell/tissue from the central or peripheral nervous system is excised and processed for protein, e.g., tissue is homogenized and protein is separated on an SDS-10% polyacylamide gel and then transferred to nitrocellulose membrane. Fluorescent protein levels are detected utilizing primary antibody/antisera (e.g., goat polyclonal raised against a particular marker protein; BD Gentest, Woburn, Mass.) and peroxidase-conjugated secondary antibody rabbit anti-goat IgG (Sigma-Aldrich). Chemiluminescence is detected using standard reagents available in the art to detect and determine levels of fluorescence marker proteins in tissue samples.

EGFP should be detected by fluorescence or by chemoluminescence specifically in remyelinating oligodendrocytes.

The present invention is not limited to the embodiments described above, but is capable of modification within the scope of the appended claims. Those skilled in the art will recognize, or be able to ascertain using no more than routine experimentation, many equivalents of the specific embodiments of the invention described herein. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for distinguishing preexisting myelinating cells from remyelinating cells upon a demyelinating insult, comprising:
(a) introducing into a population of neural cells a plurality of transgenes, wherein at least a first transgene encodes a first fluorescent marker protein and a second transgene encodes a second fluorescent marker protein, wherein said first marker protein and said second marker protein emit different detectable wavelengths; and wherein expression of said first transgene is indicative of pre-existing myelinating cells prior to induction of demyelination, and expression of said second transgene is indicative of remyelinating cells;
(b) subjecting said population of neural cells to a demyelinating insult; and
(c) identifying cells expressing said first or said second marker protein, thereby distinguishing said pre-existing myelinating cells from said remyelinating cells.

2. The method of claim 1, wherein said first transgene and said second transgene are operably linked to a regulatory element specific for myelinating cells.

3. The method of claim 1, wherein expression of said second transgene is temporally controlled by a third transgene expressed in a progenitor cell that exhibits capability to remyelinate upon said demyelinating insult.

4. The method of claim 1, wherein said second transgene is expressed when expression of said first transgene is suppressed.

5. The method of claim 4, wherein expression of said first transgene is suppressed by excising said first transgene from an expression operon.

6. A method for distinguishing pre-existing neurons from regenerated neurons upon a neural damage, comprising:
(a) introducing into a population of neuronal cells a plurality of transgenes, wherein at least a first transgene encodes a first fluorescent marker protein and a second transgene encodes a second fluorescent marker protein, wherein said first marker protein and said second marker protein emit different detectable wavelengths; and wherein expression of said first transgene is indicative of pre-existing neurons prior to a neural damage, and expression of said second transgene is indicative of regenerated neurons;
(b) subjecting said population of neuronal cells to a neural damage; and
(c) identifying neuronal cells expressing said first or said second marker protein, thereby distinguishing said pre-existing neurons from said regenerated neurons.

7. The method of claim 6, wherein said second transgene is expressed when expression of said first transgene is suppressed.

8. The method of claim 6, wherein expression of said second transgene is temporally controlled by a third transgene, wherein said third transgene is operably linked to a regulatory element inducible upon axonal damage, and wherein upon expression of the third transgene, expression of the first transgene is suppressed.

9. A vector comprising:
(a) a first transgene encoding a first marker protein, wherein expression of said first transgene is under the control of a glial cell specific regulatory element; and
(b) a second transgene encoding a second marker protein, wherein said second marker protein is expressed when expression of said first marker protein is suppressed, and wherein said first and second marker proteins are different proteins.

10. The vector of claim 9, wherein said first and second marker proteins are fluorescent and each emits a different detectable wavelength.

11. A cell comprising said vector of claim 10.

12. The cell of claim 11, wherein said cell is a neural cell.

13. A transgenic animal comprising: a first transgene encoding a first fluorescent marker protein and a second transgene encoding a second fluorescent marker protein, wherein said second marker protein is distinguishable from said first marker protein, and wherein expression of said first and said second marker protein is temporally controlled by an exogenous agent, and said expression occurs in a subpopulation of glial cells.

14. The transgenic animal of claim 13, wherein said subpopulation of glial cells are mature oligodendrocytes.

15. The transgenic animal of claim 13, wherein said subpopulation of glial cells are remyelinating oligodendrocytes.

16. The transgenic animal of claim 13, wherein said exogenous agent induces expression of a third transgene in said subpopulation of glial cells so as to temporally control expression of said first and said second marker protein.

17. The transgenic animal of claim 16, wherein expression of said first marker protein occurs in myelinating glial cells existing prior to induction by said exogenous agent, and wherein expression of said second fluorescent marker protein occurs in remyelinating glial cells upon induction by said exogenous agent.

18. The transgenic animal of claim 13, wherein said glial cell is selected from a group consisting of: astrocytes, oligodendrocytes and Schwann cells.

19. A cell of said transgenic animal of claim 13.

20. A method for determining whether remyelination has occurred in an animal, comprising the steps of:
(a) providing a transgenic animal of claim 17;
(b) administering said exogenous agent to induce expression of said third transgene;
(c) subjecting said animal to a demyelinating insult; and
(d) detecting expression of said first and/or said second marker protein, thereby determining whether remyelination has occurred.

21. A method for determining whether a candidate substance modulates remyelination comprising:
(a) providing a transgenic animal of claim 17;
(b) administering said exogenous agent to induce expression of said third transgene;
(c) subjecting said animal to a demyelination insult;
(d) exposing said animal to said candidate substance; and
(e) detecting a fluorescent signal from said first and/or said second marker protein as compared to a control, wherein a decrease in said fluorescent signal of said second marker protein after exposure to said candidate substance indicates that said substance inhibits remyelination; and wherein an increase in said fluorescent signal indicates that said candidate substance promotes remyelination.

22. A method for identifying a candidate substance for promoting remyelination comprising:
(a) providing a plurality of glial cells, at least one member of the plurality comprising a first transgene encoding a first fluorescent marker protein and a second transgene encoding a second fluorescent marker protein, wherein said second marker protein is distinguishable from said first marker protein, wherein expression of said first and said second marker protein is temporally controlled by an exogenous agent such that expression of said first marker protein occurs in myelinating glial cells existing prior to induction by said exogenous agent, and wherein expression of said second marker protein occurs in remyelinating glial cells upon induction by said exogenous agent;
(b) administering said exogenous agent;
(c) subjecting said cells to a demyelination insult;
(d) exposing said cells to a candidate substance;
(e) detecting a fluorescent signal from said first and/or said second marker protein as compared to a control, wherein a decrease in said fluorescent signal of said second marker protein after exposure to said candidate substance indicates that said substance inhibits remyelination; and wherein an increase in said fluorescent signal indicates that said candidate substance promotes remyelination

23. An animal model for detecting and quantifying remyelination comprising a double transgenic animal capable of temporally expressing a recombinase expressed in progenitor oligodendrocytes, whereby expression of said recombinase results in expression of a fluorescent marker protein, which expression corresponds to remyelination by said progenitor oligodendrocytes, and which expression provides a measure for a level of expression, thereby providing a system to both detect and quantify remyelination.


SEQUENCE LISTING (14 sequences)

SEQ ID NO: 1 - protein, 4 aa, Artificial Sequence
  Description of Artificial Sequence Synthetic peptide
KDEL

SEQ ID NO: 2 - DNA, 22 bp, Artificial Sequence
  Description of Artificial Sequence Synthetic primer
taggtgacactatacctgcagg

SEQ ID NO: 3 - DNA, 22 bp, Artificial Sequence
  Description of Artificial Sequence Synthetic primer
tacgactcactatagggagacc

SEQ ID NO: 4 - DNA, 26 bp, Artificial Sequence
  Description of Artificial Sequence Synthetic primer
tgcgaggccagaggccacttgtgtag

SEQ ID NO: 5 - DNA, 24 bp, Artificial Sequence
  Description of Artificial Sequence Synthetic primer
atgtgtcagtttcatagcctgaag

SEQ ID NO: 6 - DNA, 22 bp, Artificial Sequence
  Description of Artificial Sequence Synthetic primer
attgaggatcctggcttgactc

SEQ ID NO: 7 - DNA, 22 bp, Artificial Sequence
  Description of Artificial Sequence Synthetic primer
tattcaacttgcaccatgccgc

SEQ ID NO: 8 - DNA, 25 bp, Artificial Sequence
  Description of Artificial Sequence Synthetic primer
aatgtgcctgccttcgatctcactc

SEQ ID NO: 9 - DNA, 26 bp, Artificial Sequence
  Description of Artificial Sequence Synthetic primer
cctgcaactcacctggtacatagatg

SEQ ID NO: 10 - DNA, 23 bp, Artificial Sequence
  Description of Artificial Sequence Synthetic primer
ttaactcggtgactcagaggcag

SEQ ID NO: 11 - DNA, 25 bp, Artificial Sequence
  Description of Artificial Sequence Synthetic primer
tgtgacaaagaggccactgttgttc

SEQ ID NO: 12 - DNA, 25 bp, Artificial Sequence
  Description of Artificial Sequence Synthetic primer
acacatgcgtccttgttctcctaac

SEQ ID NO: 13 - DNA, 20 bp, Artificial Sequence
  Description of Artificial Sequence Synthetic primer
ccagaggccacttgtgtagc

SEQ ID NO: 14 - DNA, 339 bp, Artificial Sequence
  Description of Artificial Sequence Synthetic nucleotide sequence
gccacctgacgtgcggccgcacgtctaagaaaccattattatcatgacattaacctataaaaataggcgtatcacgaggccctttcgtcttcaagaattccgatcatattcaataacccttaatataacttcgtataatgtatgctatacgaagttattaggtctgaagaggagtttacgtccagccaagcttaggatcccgggtaccgatatcaagcttaggatccggaacccttaatataacttcgtataatgtatgctatacgaagttattaggtccctcgaagaggttcactagtactggccattgcggccgcttcgaggctgcctcgcgcgttt